SEQUENCE LISTING (66 sequences)

SEQ ID NO: 1 - DNA, 1599 bp, Saccharomyces cerevisiae
  source 1..1599: mol_type = genomic DNA
atgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacaccagtgggtttttcaatgatactattgatccaagacaaagatgcgttgcaatttggacttataacactcctgaaagtgaagagcaatacattagctattcccttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctcggatgacttgaagtcctggaagttagaatctgcatttgccaatgaaggtttcttaggctaccaatatgaatgtccaggtttgattgaagtcccaactgagcaagatccttccaaatcctattgggtcatgtttatttctatcaacccaggtgcacctgctggcggttccttcaaccaatattttgttggatccttcaatggtactcattttgaagcgtttgacaatcaatctagagtggtagattttggtaaggactactatgccttacaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggcccctggtctcgttttgctactaacacaactctaactaaggccaattcttacaatgtcgatttgagcaactcgactggtaccctagagtttgagttggtttacgctgttaacaccacacaaaccatatccaaatccgtcttccccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccaccggtgtcgataatttgttctacattgacaagttccaagtaagggaagtaaaatag

SEQ ID NO: 2 - DNA, 1598 bp, Saccharomyces cerevisiae
  source 1..1598: mol_type = genomic DNA
atgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacaccagtgggtttttcaatgatactattgatccaagacaaagatgcgttgcaatttggtttataacactcctgaaagtgaagagcaatacattagctattcccttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctcggatgacttgaagtcctggaagttagaatctgcatttgccaatgaaggtttcttaggctaccaatatgaatgtccaggtttgattgaagtcccaactgagcaagatccttccaaatcctattgggtcatgtttatttctatcaacccaggtgcacctgctggcggttccttcaaccaatattttgttggatccttcaatggtactcattttgaagcgtttgacaatcaatctagagtggtagattttggtaaggactactatgccttacaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggcccctggtctcgttttgctactaacacaactctaactaaggccaattcttacaatgtcgatttgagcaactcgactggtaccctagagtttgagttggtttacgctgttaacaccacacaaaccatatccaaatccgtcttccccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccaccggtgtcgataatttgttctacattgacaagttccaagtaagggaagtaaaatag

SEQ ID NO: 3 - protein, 532 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 4 - protein, 161 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWFITLLKVKSNTLAIPLMVVTLLLNTKRTLF

SEQ ID NO: 5 - DNA, 1506 bp, Saccharomyces cerevisiae
  source 1..1506: mol_type = genomic DNA
atgtcccatcgtatagcaccttccaaagaacgatcttcatcatttatttcaattttagacgatgaaacaagagacacattgaaagctaatgcagtcatggatggtgaagtagatgtcaaaaaaacaaaaggaaaaagctctcggtatatcccaccatggacaactccatatataataggtataggtggtgcttcaggttcaggcaagacaagcgttgctgctaagattgtgtcgtcaattaatgttccctggacagtattaatatctttggataacttttacaatccattaggcccagaggacagagccagagcctttaaaaatgaatacgatttcgacgagccaaatgccatcaacttagatttggcatataagtgcattttgaacttaaaggagggcaaaaggacaaatatcccagtttatagcttcgtccaccacaatagagttcctgataaaaatatagtcatatacggggccagtgtggtagttatcgaagggatctacgccctttacgatcgccgattgctggatttgatggacttgaaaatttatgttgacgctgatttggatgtctgcttagcaagaagattgtcgagagatatagtttccagagggagagatttggatggttgtattcaacaatgggagaaatttgtgaaaccaaatgcggtaaagtttgtgaaaccaacaatgaagaatgcagatgctatcattccatcgatgagtgataatgctacagcggtaaatttaatcattaaccacatcaagtcaaaactggaactaaaatcaaatgaacacttaagagagctaatcaaattgggctcttctccttcacaagatgtgcttaatcgtaacataattcatgaattgccgcccaccaaccaagttctttcgctgcatactatgcttctaaataaaaatctaaattgcgcggactttgttttctactttgacaggttagcaacaattttgttatcatgggcacttgatgacattcctgtagcacatacgaacataattacacctggtgagcataccatggaaaacgttattgcctgtcaattcgatcaagttacagctgttaatattattcgatctggcgattgttttatgaagtctttgagaaagacgatccccaatatcacaattggtaaattgttgattcagtccgattcacaaactggggaacctcaactgcattgcgaatttttacccccaaatatagaaaagtttggcaaggttttcttaatggaaggtcaaatcataagtggtgcggccatgatcatggccatccaggtgcttttagatcatggtattgatttggaaaagattagcgtggtggtttatttggccactggagttggtatccgacgtatattaaatgcatttgataacaaagtcaacatttttgctggtatgatcatctccagagaaaagttacaaaatcatcaatacaaatgggcattgaccagatttcttgattcaaagtattttggttgtgattga

SEQ ID NO: 6 - DNA, 1770 bp, Saccharomyces cerevisiae
  source 1..1770: mol_type = genomic DNA
atgactatttcttctgcacatccagagacagaaccaaagtggtggaaagaggccacgttctatcaaatttacccagcaagtttcaaagactctaatgacgatggctggggtgatatgaagggtatttcctccaagttggagtatatcaaggagcttggtgtcgatgccatttggatctcaccattctacgactcgccacaagatgatatgggttacgatattgccaactacgaaaaggtctggccaacctacggtacgaacgaggactgctttgccttgatcgaaaagacacataaacttggtatgaaatttatcaccgacttggtcatcaatcactgttccagcgaacatgaatggttcaaagagagcagatcctcgaagaccaatccgaagcgtgactggttcttctggagacctcctaagggttatgacgccgaaggcaagccaattcctccaaataattggaagtcctattttggtggttccgcatggaccttcgatgaaaagacacaagaattctacttgcgtttgttttgctccactcaacctgatttgaattgggagaatgaagactgtagaaaggcaatctacgaaagtgccgttggatactggttagaccatggtgtagacggctttagaattgatgtcggaagtttgtactccaaagttgtaggtttaccagatgcccctgttgttgacaaaaactcgacttggcaatccagtgatccatgcacattgaatggaccacgtattcacgagttccatcaagaaatgaatcaattcatcagaaacagagtgaaggatggcagggagattatgacagttggtgaaatgcaacatgcttccgacgaaactaagagactttatacgagtgcttcaagacacgaacttagtgagttatttaacttttcccacactgatgtggggacttcacctttgttccgttacaacttggtcccatttgaactgaaggattggaagattgcccttgctgagctgttcaggttcattaatggtacagattgttggtcaacaatctatctggaaaatcacgaccaacctcgttcaattacgagatttggtgacgattctcctaagaaccgtgttatttctggtaagttactctctgtgttgctaagtgccttgaccggtactctatatgtgtatcagggacaagagcttggccaaatcaatttcaagaactggcctgttgaaaagtacgaggatgtcgaaatcagaaacaactacaatgcaattaaagaagagcatggggaaaactcagaggagatgaaaaagtttttagaagccattgcccttatctccagggaccatgctagaacacctatgcaatggtctcgtgaggagccaaatgctggtttttctggtcctagtgctaaaccatggttttacttgaacgactctttcagagaaggcattaacgtcgaagatgaaatcaaggatcccaactcggttttgaacttctggaaggaggccttgaagtttagaaaggcgcataaagacattactgtgtacggatacgatttcgagtttattgatttagacaataagaagttgtttagcttcacaaagaagtacaacaataaaacattgtttgcggctttgaactttagctctgatgcgacagatttcaagattccaaatgatgattcatcgttcaagttagagtttggaaactatccaaagaaggaggtagatgcctcttccagaacattgaagccatgggaaggaagaatatatatcagcgaatga

SEQ ID NO: 7 - DNA, 1503 bp, Saccharomyces cerevisiae
  source 1..1503: mol_type = genomic DNA
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgatgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacggaagtcggtaagagtgttgctgtcgactcttctgaatctaacttgaagaaaatcactttggaactaggtggtaagtccgcccatttggtctttgacgatgctaacattaagaagactttaccaaatctagtaaacggtatcttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 8 - DNA, 1503 bp, Saccharomyces cerevisiae
  source 1..1503: mol_type = genomic DNA
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccacccctgaagatgttgaatatgctatcgaatgtgccgaccgtgctttccacgacactgaatgggctacccaagacccaagagaaagaggccgtctactaagtaagttggctgatgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacggaagtcggtaagagtgttgctgtcgactcttctgaatctaacttgaagaaaatcactttggaactaggtggtaagtccgcccatttggtctttgacgatgctaacattaagaagactttaccaaatctagtaaacggtatcttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 9 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 10 - protein, 500 aa, Saccharomyces cerevisiae
MTKLHFDTAEPVKITLPNGLTYEQPTGLFINNKFMKAQDGKTYPVEDPSTENTVCEVSSATPEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 11 - DNA, 74 bp, synthetic construct
  source 1..74: mol_type = other DNA
gaagaaatacgcgtagcgttaatcgaccccacgtccagggtttttccatgcgggcgaatttctgtcgagtcatg

SEQ ID NO: 12 - DNA, 68 bp, synthetic construct
  source 1..68: mol_type = other DNA
agaatggcttttgaaaaaaataaaaaagacaataagttttataacctctagcggccgcatcaagcttg

SEQ ID NO: 13 - DNA, 74 bp, synthetic construct
  source 1..74: mol_type = other DNA
tttgtagggtttcttcgcacattatcattattattctttgagaatactcacgggcgaatttctgtcgagtcatg

SEQ ID NO: 14 - DNA, 68 bp, synthetic construct
  source 1..68: mol_type = other DNA
atcaaacaagatacaaacaaagcttttcaacgtaatatttactatcgatggcggccgcatcaagcttg

SEQ ID NO: 15 - DNA, 68 bp, synthetic construct
  source 1..68: mol_type = other DNA
tcgatgccatttggatctcaccattctacgactcgccacaagatgatatggcggccgcatcaagcttg

SEQ ID NO: 16 - DNA, 74 bp, synthetic construct
  source 1..74: mol_type = other DNA
atcaagaaacatctttaacatacacaaacacatactatcagaatacaatgcgggcgaatttctgtcgagtcatg

SEQ ID NO: 17 - DNA, 68 bp, synthetic construct
  source 1..68: mol_type = other DNA
atgaaagtattttgtgtatatgacggaaagaaatgcaggttggtacattagcggccgcatcaagcttg

SEQ ID NO: 18 - DNA, 74 bp, synthetic construct
  source 1..74: mol_type = other DNA
gtatgaaggatacacaaaaaaaataaagattaagaaagttattcaaaatgcgggcgaatttctgtcgagtcatg

SEQ ID NO: 19 - DNA, 75 bp, synthetic construct
  source 1..75: mol_type = other DNA
agattaatagttcttaaaagagataaataaatcatttaattaaaaaactgatttttagcggccgcatcaagcttg

SEQ ID NO: 20 - DNA, 74 bp, synthetic construct
  source 1..74: mol_type = other DNA
gataatttcatacgtttaatttcgaactcgcatttattttatttattatgcgggcgaatttctgtcgagtcatg

SEQ ID NO: 21 - DNA, 68 bp, synthetic construct
  source 1..68: mol_type = other DNA
tacgtgcactattatttaattttactttatattgcctctaattattctcagcggccgcatcaagcttg

SEQ ID NO: 22 - DNA, 26 bp, synthetic construct
  source 1..26: mol_type = other DNA
gatcggcaagctttccttttcctttg

SEQ ID NO: 23 - DNA, 26 bp, synthetic construct
  source 1..26: mol_type = other DNA
aaaacaaaggaaaaggaaagcttgcc

SEQ ID NO: 24 - DNA, 26 bp, synthetic construct
  source 1..26: mol_type = other DNA
gatcgatgtattgctcttcactttcg

SEQ ID NO: 25 - DNA, 26 bp, synthetic construct
  source 1..26: mol_type = other DNA
aaaacgaaagtgaagagcaatacatc

SEQ ID NO: 26 - DNA, 26 bp, synthetic construct
  source 1..26: mol_type = other DNA
gatcgcactgaaaacaccgtttgtgg

SEQ ID NO: 27 - DNA, 26 bp, synthetic construct
  source 1..26: mol_type = other DNA
aaaaccacaaacggtgttttcagtgc

SEQ ID NO: 28 - DNA, 26 bp, synthetic construct
  source 1..26: mol_type = other DNA
gatcgacctttgttccgttacaactg

SEQ ID NO: 29 - DNA, 26 bp, synthetic construct
  source 1..26: mol_type = other DNA
aaaacagttgtaacggaacaaaggtc

SEQ ID NO: 30 - DNA, 26 bp, synthetic construct
  source 1..26: mol_type = other DNA
gatcgtcacaatgtcacagtctcaag

SEQ ID NO: 31 - DNA, 26 bp, synthetic construct
  source 1..26: mol_type = other DNA
aaaacttgagactgtgacattgtgac

SEQ ID NO: 32 - DNA, 26 bp, synthetic construct
  source 1..26: mol_type = other DNA
gatcgagtcatatacggggccagtgg

SEQ ID NO: 33 - DNA, 26 bp, synthetic construct
  source 1..26: mol_type = other DNA
aaaaccactggccccgtatatgactc

SEQ ID NO: 34 - DNA, 26 bp, synthetic construct
  source 1..26: mol_type = other DNA
gatcgcaaatgcatttaatatacgtg

SEQ ID NO: 35 - DNA, 26 bp, synthetic construct
  source 1..26: mol_type = other DNA
aaaacacgtatattaaatgcatttgc

SEQ ID NO: 36 - DNA, 60 bp, synthetic construct
  source 1..60: mol_type = other DNA
acaagcaaaacaaaaagcttttcttttcactaacgtatatgatgcttttgcaagctttcc

SEQ ID NO: 37 - DNA, 60 bp, synthetic construct
  source 1..60: mol_type = other DNA
acaagcaaaacaaaaagcttttcttttcactaacgcatatgatgcttttgcaagctttcc

SEQ ID NO: 38 - DNA, 60 bp, synthetic construct
  source 1..60: mol_type = other DNA
gatgcagatattttggctgcaaaaccagctaaaaggaaaaggaaagcttgcaaaagcatc

SEQ ID NO: 39 - DNA, 60 bp, synthetic construct
  source 1..60: mol_type = other DNA
ttgatccaagacaaagatgcgttgcgatttggacttataacactcctgaaagtgaggaac

SEQ ID NO: 40 - DNA, 59 bp, synthetic construct
  source 1..59: mol_type = other DNA
ttgatccaagacaaagatgcgttgcgatttggtttataacactcctgaaagtgaggaac

SEQ ID NO: 41 - DNA, 60 bp, synthetic construct
  source 1..60: mol_type = other DNA
gtaaaagtgtaaccaccatcaagagaatagctaatatactgttcctcactttcaggagtg

SEQ ID NO: 42 - DNA, 80 bp, synthetic construct
  source 1..80: mol_type = other DNA
tccagatagattgttgaccaacaatctgtaccattaatgaacctgaacagctcagcaagggcaatcttccaatccttcag

SEQ ID NO: 43 - DNA, 80 bp, synthetic construct
  source 1..80: mol_type = other DNA
tccagatagattgttgaccaacaatctgtaccattaatgtacctgaacagctcagcaagggcaatcttccaatccttcag

SEQ ID NO: 44 - DNA, 60 bp, synthetic construct
  source 1..60: mol_type = other DNA
gacttcacctttgttccgttacaacttagtcccatttgaactgaaggattggaagattgc

SEQ ID NO: 45 - DNA, 60 bp, synthetic construct
  source 1..60: mol_type = other DNA
gaaagccatttttaatgagttatatagcgtcgttgattaggtatcgtatcacaatgtcac

SEQ ID NO: 46 - DNA, 60 bp, synthetic construct
  source 1..60: mol_type = other DNA
gaaagccatttttaatgagttatatagggtcgttgattaggtatcgtatcacaatgtcac

SEQ ID NO: 47 - DNA, 60 bp, synthetic construct
  source 1..60: mol_type = other DNA
actggaaggagtgatggttgatgtattttctcttgagactgtgacattgtgatacgatac

SEQ ID NO: 48 - DNA, 60 bp, synthetic construct
  source 1..60: mol_type = other DNA
cgatagcatattcaacatcttcaggggtggcagaagagacttcacaaacggtgttttcag

SEQ ID NO: 49 - DNA, 60 bp, synthetic construct
  source 1..60: mol_type = other DNA
cgatagcatattcaacatcttcagtggtggcagaagagacttcacaaacggtgttttcag

SEQ ID NO: 50 - DNA, 60 bp, synthetic construct
  source 1..60: mol_type = other DNA
gctcaagacggtaagacctatcccgtcgaagatccttccactgaaaacaccgtttgtgaa

SEQ ID NO: 51 - DNA, 80 bp, synthetic construct
  source 1..80: mol_type = other DNA
attttgaacttaaaggagggcaaaaggacaaatatcccagtttatagcttcgtccaccacaatagagttcctgataaaaa

SEQ ID NO: 52 - DNA, 80 bp, synthetic construct
  source 1..80: mol_type = other DNA
attttgaacttaaaggagggcaaaaggacaaatctcccagtttatagcttcgtccaccacaatagagttcctgataaaaa

SEQ ID NO: 53 - DNA, 80 bp, synthetic construct
  source 1..80: mol_type = other DNA
cgatcgtaaagggcgtagatcccttcgataactactacactggccccgtatatgactatatttttatcaggaactctatt

SEQ ID NO: 54 - DNA, 60 bp, synthetic construct
  source 1..60: mol_type = other DNA
ttagcgtggtggtttatttggccactggagttggtatcagacgtatattaaatgcatttg

SEQ ID NO: 55 - DNA, 60 bp, synthetic construct
  source 1..60: mol_type = other DNA
ttagcgtggtggtttatttggccactgaagttggtatcagacgtatattaaatgcatttg

SEQ ID NO: 56 - DNA, 60 bp, synthetic construct
  source 1..60: mol_type = other DNA
ctggagatgatcataccagcaaaaatgttgactttgttatcaaatgcatttaatatacgt

SEQ ID NO: 57 - DNA, 20 bp, synthetic construct
  source 1..20: mol_type = other DNA
gcctttgttgaactcgatcc

SEQ ID NO: 58 - DNA, 25 bp, synthetic construct
  source 1..25: mol_type = other DNA
cataaagttttacattcgtcactcg

SEQ ID NO: 59 - DNA, 19 bp, synthetic construct
  source 1..19: mol_type = other DNA
agtatctacggcgcagtac

SEQ ID NO: 60 - DNA, 26 bp, synthetic construct
  source 1..26: mol_type = other DNA
cagatcaaacaagatacaaacaaagc

SEQ ID NO: 61 - DNA, 25 bp, synthetic construct
  source 1..25: mol_type = other DNA
gtttggtaatattcaattcgaagtg

SEQ ID NO: 62 - DNA, 19 bp, synthetic construct
  source 1..19: mol_type = other DNA
ggctgatgaattggaaagc

SEQ ID NO: 63 - DNA, 23 bp, synthetic construct
  source 1..23: mol_type = other DNA
ttccataaagcattacttctgcg

SEQ ID NO: 64 - DNA, 21 bp, synthetic construct
  source 1..21: mol_type = other DNA
gcgtgcatgtatcgtagtaac

SEQ ID NO: 65 - DNA, 18 bp, synthetic construct
  source 1..18: mol_type = other DNA
gagaggtgtaccagccag

SEQ ID NO: 66 - DNA, 23 bp, synthetic construct
  source 1..23: mol_type = other DNA
ccacttgttctcactatttcctc

(19) United States
(12) Patent Application Publication
(10) Pub. No.: US 2024/0309313 A1
(43) Pub. Date: Sep. 19, 2024

(54) Yeast Single Nucleotide Polymorphisms for Industrially Relevant Phenotypes

(71) Applicants: VIB VZW, Gent (BE); Katholieke Universiteit Leuven, Leuven (BE); THE BOARD OF TRUSTEES OF THE LELAND STANFORD JUNIOR UNIVERSITY, Stanford, CA (US)

(72) Inventors: Kevin Verstrepen, Leuven (BE); Ping-Wei Ho, Leuven (BE); Dan Jarosz, Stanford, CA (US)

(21) Appl. No.: 18/577,903

(22) PCT Filed: Jul. 7, 2022
(86) PCT No.: PCT/EP2022/068897

§ 371 Date: Jan. 9, 2024

Related U.S. Application Data
(60) Provisional application No. 63/219,993, filed on Jul. 9, 2021.

(51) Int. Cl. C12N 1/18; A61K 36/064; C12N 9/22; C12N 15/11; C12N 15/81; C12P 7/16; C12P 7/54; C12R 1/865

(52) U.S. Cl. CPC C12N 1/185; A61K 36/064; C12N 9/22; C12N 15/111; C12N 15/81; C12P 7/16; C12P 7/54; C12N 2310/20; C12R 2001/865

(57) ABSTRACT

The present invention relates to the field of yeast fermentations. More particularly, the invention relates to mutant alleles useful to engineer industrially relevant traits in yeast.

### CROSS REFERENCE TO RELATED APPLICATIONS

This application is a national phase entry under 35 U.S.C. § 371 of International Patent Application PCT/EP2022/068425, filed Jul. 4, 2022, designating the United States of America and published in English as International Patent Publication WO 2023/280766 on Jan. 12, 2023, which claims the benefit under Article 8 of the Patent Cooperation Treaty to European Patent Application Serial No. 21184332.1, filed Jul. 7, 2021, the entireties of which are hereby incorporated by reference.

### INCORPORATION BY REFERENCE

The ST.26 XML Sequence listing named “10312US20230108SequenceListingST26”, created on Jun. 29, 2022, and having a size of 71,696 bytes, is hereby incorporated herein by this reference in its entirety.

### FIELD OF THE INVENTION

The present invention relates to the field of yeast fermentations. More particularly, the invention relates to mutant alleles useful to engineer industrially relevant traits in yeast.

### BACKGROUND

The brewer's yeast Saccharomyces cerevisiae is exploited in several industrial processes, ranging from food and beverage fermentation to the production of biofuels, pharmaceuticals and complex chemicals. There is an increasing demand from industry for strains that show increased fermentation efficiency, stress resistance, substrate range or even specific aroma profiles. However, despite the enormous genetic and phenotypic diversity within this species, genomics and metagenomics studies revealed that the set of S. cerevisiae strains currently used in industrial settings only represent a small fraction of the existing natural diversity (Liti et al., 2009; Fay & Benavides, 2005; Carreto et al., 2008; Gallone et al., 2016; Gallone et al., 2019).

### SUMMARY

Here, we combined the power of 1,125 fully sequenced inbred segregants with high-throughput phenotyping methods to identify 4 mutant alleles relevant to industrial fermentation processes.

One aspect of the invention is an industrial yeast strain comprising a homozygous or hemizygous disrupted, partially deleted or completely deleted ALD6 allele. In one embodiment, said ALD6 allele encodes an Ald6 protein comprising a T to P mutation on position 62 of SEQ ID No. 9, more particularly, said ALD6 allele encodes SEQ ID No. 10. The application also provides a chimeric gene construct comprising a promoter active in yeast operably linked to a Crispr guide RNA targeting an ALD6 allele, as well as an industrial yeast strain comprising said chimeric gene construct.

The application also provides the use of a genetic inhibitor of ALD6 to develop a yeast strain with an increased production of acetic acid and the use of said genetic inhibitor or of a yeast strain comprising said genetic inhibitor to increase the acetic acid production in a yeast fermentation. In a particular embodiment, said use is provided, wherein the genetic inhibitor is a disrupted, partially deleted or completely deleted ALD6 allele, a nuclease, a Crispr-Cas effector, the chimeric gene construct of claim 7 or an RNA-silencing agent.

In another aspect, the application provides a S. cerevisiae var. boulardii comprising a homozygous or hemizygous disrupted, partially deleted or completely deleted ALD6 allele for use as a medicine, more particularly for use in the treatment or prevention of gastrointestinal disorders, diarrhea, gastrointestinal discomfort and/or constipation. Also a food or feed product, beverage, food supplement, dietary supplement or pharmaceutical composition comprising said S. cerevisiae var. boulardii is provided.

In another aspect, a chimeric gene construct is provided comprising a promoter active in yeast operably linked to a Crispr guide RNA targeting a SUC2, IMA1 or URK1 allele. An industrial yeast strain comprising said chimeric gene construct or comprising a homozygous or hemizygous disrupted, partially deleted or completely deleted SUC2, IMA1 or URK1 allele is also provided.

In one embodiment, the use of a genetic inhibitor of SUC2 to develop a yeast strain with a reduced production of ethanol, 1-propanol, ethyl acetate, acetic acid and/or glycerol is provided, as well as the use of said genetic inhibitor of SUC2 or of an industrial yeast comprising said genetic inhibitor for reducing the production of ethanol, 1-propanol, ethyl acetate, acetic acid and/or glycerol in a yeast fermentation. Said SUC2 genetic inhibitor can be a disrupted, partially deleted or completely deleted SUC2, a SUC2 allele encoding SEQ ID No. 4, a nuclease, a Crispr-Cas effector, the chimeric gene construct comprising a promoter active in yeast operably linked to a Crispr guide RNA targeting SUC2, or an RNA-silencing agent.

In another aspect, the use is provided of a genetic inhibitor of IMA1 to develop a yeast strain with a reduced production of glycerol and/or acetic acid or with an increased production of isobutanol. Also the use of said genetic inhibitor of IMA1 or of an industrial yeast comprising said inhibitor is provided for reducing the production of glycerol and/or acetic acid or for increasing the production of isobutanol in a yeast fermentation. Said IMA1 genetic inhibitor can be a disrupted, partially deleted or completely deleted IMA1 allele, a nuclease, a Crispr-Cas effector, the chimeric gene construct comprising a promoter active in yeast operably linked to a Crispr guide RNA targeting IMA1, or an RNA-silencing agent.

In yet another aspect, the use is provided of a genetic inhibitor of URK1 to develop a yeast strain with an increased production of isobutanol. Also the use is provided of said genetic inhibitor or of an industrial yeast comprising said inhibitor for increasing the production of isobutanol in a yeast fermentation. Said URK1 genetic inhibitor is a disrupted, partially deleted or completely deleted URK1 allele, a nuclease, a Crispr-Cas effector, the chimeric gene comprising a promoter active in yeast operably linked to a Crispr guide RNA targeting URK1, or an RNA-silencing agent.

### DESCRIPTION OF THE FIGURES

FIG. 1 illustrates the five candidate loci at single-nucleotide resolution identified by genome-wide mapping and predicted to affect multiple industrially-relevant phenotypes. PVAL (−log 10 transformed p-value from the forward selection) and the chromosomal position are shown for the variants centered around each candidate QTL (indicated in the grey rectangle). The threshold for selecting candidate variants is shown as the dotted line (PVAL=5.2).

FIG. 2 summarizes the effect of deleting genes that overlap with predicted QTLs on the mapped traits in both haploid strains RM11-1a (RM) and YJM975α (YJM). The trait value of the wild-type strain is set at 1. Each point is represented as the mean±STD of at least three biological replicates after normalization against the mean of the respective wild type strain for every phenotype (dotted line). P-values are indicated by asterisk symbols; *: p<=0.05, **: p<=0.01, ***: p<=0.001, ****: p<=0.0001.

FIGS. 3A-3E shows the identification of the causal variant of reduced ethanol production in the SUC2 locus. FIG. 3A) Candidate variants in SUC2. FIG. 3B) Meiotic crossovers within the SUC2 locus in the F6 segregants. Swapping the intergenic variant (−6) yields minor phenotypic effect, whereas swapping the true causal variant (394) yields the same major effect as swapping the entire haplotype block. FIG. 3C) Ethanol concentration at the end of fermentation (160P) of the wild type (WT) strain RM11-a and YJM975α and the respective variant-swapped mutants. FIG. 3D) Ethanol concentration at the end of fermentation (160P) of the wild type strain S. boulardii, Ethanol Red and CEN.PK and the 394 frameshift variant mutant. FIG. 3E) Interaction network of SUC2 and genes whose coding sequence are altered by variants that were identified for ethanol concentration phenotype. The thickness of the edges represents the confidence score associated with the interaction as determined by STRING. Data is shown with mean±STD; P-values are indicated with the level of significance (ns: not significant, *: p<=0.05, *: p<=0.01, *: p<=0.001, *: p<=0.0001).

FIG. 4 show the effect of swapping predicted QTL alleles on various phenotypes between the haploid strains RM11-1a (RM) and YJM975α (YJM). A) frameshift variant (394ΔAfs) in SUC2 and B) 184A>C in ALD6. Each point is represented as normalized mean±STD of at least three biological replicates. P-values are indicated by asterisk symbols (*: p<=0.05, **: p<=0.01, *: p<=0.001, ****: p<=0.0001).

FIGS. 5A-5D shows the identification of the causal variant underlying differences in acetic acid production FIG. 5A) Candidate variants in ALD6. FIG. 5B) Meiotic crossovers within the ALD6 locus in the F6 segregants. FIG. 5C) Acetic acid production of the variants swapped in the parent strain RM11-a and YJM975α, and FIG. 5D) in S. boulardii, Ethanol Red, and CEN.PK. Error bars represent standard deviation of 3 biological replicates. P-values are indicated with the level of significance (*: p<=0.05, **: p<=0.01, ***: p<=0.001, ****: p<=0.0001).

FIG. 6 shows isobutanol production of URK1 knockout (KO) and WT strains of Ethanol Red and CEN.PK. Error bars represent standard deviations from three biological replicates. P-values are indicated by asterisk symbols (*: p<=0.05, *: p<=0.01, *: p<=0.001, *: p<=0.0001).

### DETAILED DESCRIPTION

The inventors of current application performed a series of experiments aiming at obtaining a comprehensive view on the different QTL loci that contribute to industrially relevant properties in S. cerevisiae. To this end, 1125 F6 segregants were obtained from a cross of two phenotypically divergent S. cerevisiae strains, RM11-1a, a natural vineyard isolate, and YJM975α, originally isolated from an immunocompromised patient in Italy (She & Jarosz, 2018). For each of the 1125 segregants and the two parental yeasts, we set up a fermentation reaction in medium and conditions mimicking industrial wort fermentation. For each of these 1125 fermentations, we measured 18 different industrially-relevant parameters, including the production of primary and secondary metabolites. In addition, we also screened all 1125 segregants for their resistance to various stress factors and consumption of maltose. This large set of phenotypic data was subsequently combined with the available genome sequences of each of the segregants and analyzed using the pipeline developed by She & Jarosz (2018) to identify QTLs underlying the phenotypes (see methods). Finally, some of the QTLs were experimentally confirmed to verify the mapping and explore the possibilities to use the data to engineer superior industrial yeasts.

Experimental validation confirmed the contribution of four genetic loci, of which two were pinpointed to the single-nucleotide level to a plethora of industrially relevant traits. The identified loci consisted of both coding and intergenic regions, and comprised a broad range of different types of mutations, ranging from structural variation to InDels (inserts/deletions) and SNPs (single nucleotide polymorphisms). Interestingly, many of the loci were predicted and validated for multiple phenotypes, indicating that QTLs often affect multiple phenotypes. Importantly, the inventors of current application were able to reproduce the effect of some QTLs in other industrial yeast strains. For example, transferring the ALD6^{184A>C} mutation in various strains invariably led to increased acetic acid formation, while deletion of URK1 led to increased isobutanol production. These results demonstrate the effectivity of the current approach to detecting causal variants for complex traits and open new avenues for optimizing strains in a broad range of biotechnological applications. Based hereon, the invention is defined in the following aspects and embodiments.

A SUC2 Allele Providing Reduced Production of Ethanol, 1-Propanol, Ethyl Acetate, Acetic Acid and/or Glycerol

In a first aspect, a yeast strain is provided comprising a disrupted, partially deleted or completely deleted SUC2 allele. This is equivalent as saying that a yeast strain is provided comprising a deficient SUC2 allele.

SUC2 (YIL162W; SGD:S000001424) encodes for Invertase 2 (EC:3.2.1.26). Alternative names are beta-fructofuranosidase 2 and saccharase. The wild-type DNA sequence is depicted in SEQ ID No. 1 and the wild-type protein sequence in SEQ ID No. 3.

In one embodiment, the yeast strain comprising a disrupted, partially deleted or completely deleted SUC2 allele is a haploid yeast strain or a haploid segregant from a diploid yeast. In another embodiment, said yeast strain is a diploid yeast strain. In yet another embodiment, said yeast strain is a diploid yeast strain and the disrupted, partially deleted or completely deleted SUC2 allele is present in homozygous form, meaning that both SUC2 alleles of said diploid yeast strain are a disrupted, partially deleted or completely deleted SUC2 allele. In a further embodiment, both SUC2 alleles are identical. In another further embodiment, both SUC2 alleles are different.

In another embodiment, a yeast strain is provided comprising a homozygous or hemizygous mutant SUC2 allele wherein said SUC2 allele compromises, partially abolishes or completely abolishes Suc2 function. A compromised, partially abolished or completely abolished Suc2 function can easily be checked by the skilled person by measuring the residual sucrose level in a finished fermentation sample by standard techniques.

In a particular embodiment, said disrupted, partially deleted or completely deleted SUC2 allele encodes a truncated Suc2 protein, more particularly a C-terminally truncated Suc2 protein, even more particularly a C-terminally truncated Suc2 protein lacking the at least 50, 100, 150, 200, 250, 300, 350, 360, 370, 380, 390, 400, or 401 most C-terminal amino acids of SEQ ID No. 3. In another particular embodiment, said disrupted, partially deleted or completely deleted SUC2 allele comprises a frameshift mutation at position 394 of SEQ ID No. 1. Even more particularly, said SUC2 allele is the SUC2 allele encoding SEQ ID No. 4 or as depicted in SEQ ID No. 2.

In another particular embodiment, said yeast strain of the first aspect and any of its embodiments is an industrial yeast strain. In another particular embodiment, said yeast strain of the first aspect and any of its embodiments is a Saccharomyces cerevisiae strain. Even more particularly, said yeast strain is not S. cerevisiae YJM975, YJM969, YJM1332 or YJM981. In yet another particular embodiment, said yeast strain or said S. cerevisiae strain is an engineered or recombinant yeast or S. cerevisiae strain.

Current application further provides the use of any of the yeast strains of the first aspect and its embodiments, for reducing the production of ethanol, 1-propanol, ethyl acetate, acetic acid and/or glycerol in a yeast fermentation.

In a further embodiment also methods of statistically significantly reducing the production of ethanol, 1-propanol, ethyl acetate, acetic acid and/or glycerol in a yeast fermentation are provided.

These methods comprise the step of adding any of the yeast strains of the first aspect and its embodiments to a fermentation medium. The methods optionally further comprise the step of measuring the level of ethanol, 1-propanol, ethyl acetate, acetic acid and/or glycerol.

“Reducing” as used here refers to statistically significantly reducing the production of any of ethanol, 1-propanol, ethyl acetate, acetic acid and/or glycerol compared to the production of any of ethanol, 1-propanol, ethyl acetate, acetic acid and/or glycerol by a yeast strain comprising a wild-type or functional version of SUC2. An example of such wild-type or functional SUC2 allele is provided in SEQ ID No. 1, encoding a wild-type or functional Suc2 protein as depicted in SEQ ID No. 3.

The application also provides the use of a genetic inhibitor of SUC2 to develop a yeast strain with statistically significantly reduced production of ethanol, 1-propanol, ethyl acetate, acetic acid and/or glycerol. Said reduced production is compared to a control or an isogenic yeast strain not comprising or not treated with the genetic inhibitor. In one embodiment, said genetic inhibitor is an inhibitor of the RNA interference technology or antisense technology or alternatively phrased is an RNA-silencing agent. Non-limiting examples of RNA-silencing agents are siRNA, shRNA, dsRNA, divalent siRNA (di-siRNA), antisense oligonucleotides (ASO), gapmer, microRNA, ribozyme, DNAzyme, nucleic acid aptamer, locked nucleic acid (LNA), bridged nucleic acid (BNA), ethyl bridged nucleic acid (ENA), peptide nucleic acid (PNA) or a morpholino oligonucleotide.

In another embodiment, said genetic inhibitor is a nuclease, more particularly a CRISPR-Cas, a TALEN, a meganuclease or a Zinc-finger nuclease. The application thus also provides a chimeric gene construct comprising a promoter active in yeast operably linked to a Crispr guide RNA targeting a SUC2 allele. A non-limiting example of said Crispr guide RNA targeting SUC2 is provided in the application, however given the state of the art of the Crispr technology and the available SUC2 sequence the skilled person would have no problem in selecting alternative guide RNAs for efficiently inhibiting the Suc2 function. In one particular embodiment, a yeast strain or an industrial yeast strain is provided comprising a chimeric gene construct comprising a promoter active said yeast strain operably linked to a Crispr guide RNA targeting SUC2 or targeting a SUC2 allele, wherein the expression of SUC2 in said yeast is statistically significantly reduced compared to that in a control yeast not comprising said chimeric gene construct. This thus means that the chimeric gene construct is responsible for the reduced expression of SUC2.

In yet another embodiment, the use of a genetic inhibitor of SUC2 to develop a yeast strain with statistically significantly reduced production of ethanol, 1-propanol, ethyl acetate, acetic acid and/or glycerol is provided, wherein said genetic inhibitor is a disrupted, partially deleted or completely deleted SUC2 allele. More particularly, the SUC2 allele encoding SEQ ID No. 4 or depicted in SEQ ID No. 2. The “reduced production” as used herein is compared to a control yeast strain comprising a functional or wild-type SUC2 allele.

### An IMA1 Allele Providing Reduced Production of Glycerol and Acetic Acid and Increased Production of Isobutanol

In a second aspect, a yeast strain is provided comprising a disrupted, partially deleted or completely deleted IMA1 allele. This is equivalent as saying that a yeast strain is provided comprising a deficient IMA1 allele.

IMA1 (YGR287C; SGD:S000003519) is a member of the IMA isomaltase family and encodes oligo-1,6-glucosidase (IsoMaltase or alpha-1,6-glucosidase/alpha-methylglucosidase). Ima1 is required for isomaltose utilization and preferentially hydrolyzes isomaltose, palatinose, and methyl-alpha-glucoside, with little activity towards isomaltotriose or longer oligosaccharides. Ima1 does not hydrolyze maltose. The wild-type DNA sequence is depicted in SEQ ID No. 6.

In one embodiment, the yeast strain comprising a disrupted, partially deleted or completely deleted IMA1 allele is a haploid yeast strain or a haploid segregant from a diploid yeast. In another embodiment, said yeast strain is a diploid yeast strain. In yet another embodiment, said yeast strain is a diploid yeast strain and the IMA1 allele is present in homozygous form, meaning that both IMA1 alleles of said diploid yeast strain are a disrupted, partially deleted or completely deleted IMA1 allele. In a further embodiment, both IMA1 alleles are identical. In another further embodiment, both IMA1 alleles are different. In another embodiment, a yeast strain is provided comprising a homozygous or hemizygous mutant IMA1 allele wherein said IMA1 allele compromises, partially abolishes or completely abolishes Ima1 function.

In another particular embodiment, said yeast strain of the second aspect and any of its embodiments is an industrial yeast strain. In another particular embodiment, said yeast strain of the second aspect and any of its embodiments is a Saccharomyces cerevisiae strain. In yet another particular embodiment, said yeast strain or said S. cerevisiae strain is an engineered or recombinant yeast or S. cerevisiae strain.

Current application further provides the use of any of the yeast strains of the second aspect and its embodiments, for reducing the production of glycerol and/or acetic acid or for increasing the production of isobutanol in a yeast fermentation.

In a further embodiment also methods of statistically significantly reducing the production of glycerol and/or acetic acid or for statistically significantly increasing the production of isobutanol in a yeast fermentation are provided. These methods comprise the step of adding any of the yeast strains of the second aspect and its embodiments to a fermentation medium. The methods optionally further comprise the step of measuring the level of glycerol, acetic acid and/or isobutanol.

“Reducing” as used here refers to statistically significantly reducing the production of glycerol and/or acetic acid compared to the production of glycerol and/or acetic acid by a yeast strain comprising a wild-type or functional version of IMA1.

“Increasing” as used here refers to statistically significantly increasing the production of isobutanol compared to the production of isobutanol by a yeast strain comprising a wild-type or functional version of IMA1. An example of such wild-type or functional IMA1 allele is provided in SEQ ID No. 6.

The application also provides the use of a genetic inhibitor of IMA1 to develop a yeast strain with statistically significantly reduced production of acetic acid and/or glycerol and/or with statistically significantly increased production of isobutanol. Said reduced or increased production is compared to a control or an isogenic yeast strain not comprising or not treated with the genetic inhibitor. In one embodiment, said genetic inhibitor is an inhibitor of the RNA interference technology or antisense technology or alternatively phrased is an RNA-silencing agent. Non-limiting examples of RNA-silencing agents are siRNA, shRNA, dsRNA, divalent siRNA (di-siRNA), antisense oligonucleotides (ASO), gapmer, microRNA, ribozyme, DNAzyme, nucleic acid aptamer, locked nucleic acid (LNA), bridged nucleic acid (BNA), ethyl bridged nucleic acid (ENA), peptide nucleic acid (PNA) or a morpholino oligonucleotide.

In another embodiment, said genetic inhibitor is a nuclease, more particularly a CRISPR-Cas, a TALEN, a meganuclease or a Zinc-finger nuclease. The application thus also provides a chimeric gene construct comprising a promoter active in yeast operably linked to a Crispr guide RNA targeting an IMA1 allele. A non-limiting example of said Crispr guide RNA targeting IMA1 is provided in the application, however given the state of the art of the Crispr technology and the available IMA1 sequence the skilled person would have no problem in selecting alternative guide RNAs for efficiently inhibiting the Ima1 function. In one particular embodiment, a yeast strain or an industrial yeast strain is provided comprising a chimeric gene construct comprising a promoter active said yeast strain operably linked to a Crispr guide RNA targeting IMA1 or targeting an IMA1 allele, wherein the expression of IMA1 in said yeast is statistically significantly reduced compared to that in a control yeast not comprising said chimeric gene construct. This thus means that the chimeric gene construct is responsible for the reduced expression of IMA1.

In yet another embodiment, the use of a genetic inhibitor of IMA1 to develop a yeast strain with statistically significantly reduced production of acetic acid and/or glycerol and/or with statistically significantly increased production of isobutanol is provided, wherein said genetic inhibitor is a disrupted, partially deleted or completely deleted IMA1 allele. Said reduced or increased production is compared to a control yeast strain comprising a functional or wild-type IMA1 allele.

### An ALD6 Allele for Increased Production of Acetic Acid

In a third aspect, a yeast strain is provided comprising a disrupted, partially deleted or completely deleted ALD6 allele. This is equivalent as saying that a yeast strain is provided comprising a deficient ALD6 allele.

ALD6 (YPL061W; SGD:S000005982) is a cytosolic aldehyde dehydrogenase required for the conversion of acetaldehyde to acetate. The wild-type DNA sequence is depicted in SEQ ID No. 7 and the wild-type protein sequence in SEQ ID No. 9.

In one embodiment, the yeast strain comprising a disrupted, partially deleted or completely deleted ALD6 allele is a haploid yeast strain or a haploid segregant from a diploid yeast. In another embodiment, said yeast strain is a diploid yeast strain. In yet another embodiment, said yeast strain is a diploid yeast strain and the ALD6 allele is present in homozygous form, meaning that both ALD6 alleles of said diploid yeast strain are a disrupted, partially deleted or completely deleted ALD6 allele.

In a further embodiment, both ALD6 alleles are identical. In another further embodiment, both ALD6 alleles are different. In another embodiment, a yeast strain is provided comprising a homozygous or hemizygous mutant ALD6 allele wherein said ALD6 allele compromises, partially abolishes or completely abolishes Ald6 function.

In a particular embodiment, said disrupted, partially deleted or completely deleted ALD6 allele comprises an A to C mutation on nucleic acid position 184 of SEQ ID No. 7. This means that the adenosine at position 184 of SEQ ID No. 7 is replace by a cytosine, or alternatively phrased a A184C mutation. In another particular embodiment, said disrupted, partially deleted or completely deleted ALD6 allele encodes an Ald6 protein comprising a threonine (T) to proline (P) mutation on position 62 of SEQ ID No 9. More particularly, said Ald6 protein is the protein as depicted in SEQ ID No. 10. Even more particularly, said disrupted, partially deleted or completely deleted ALD6 allele is the ALD6 allele encoding SEQ ID No. 10 or as depicted in SEQ ID No. 8.

In another particular embodiment, said yeast strain of the third aspect and any of its embodiments is an industrial yeast strain. In another particular embodiment, said yeast strain of the third aspect and any of its embodiments is a Saccharomyces cerevisiae strain, even more particularly a CEN.PK yeast strain, an Ethanol Red yeast strain or a S. cerevisiae var. boulardii yeast strain. In yet another particular embodiment, said yeast strain or said S. cerevisiae strain is an engineered or recombinant yeast or S. cerevisiae strain. Even more particularly, said yeast strain is not S. cerevisiae HB_C_TUKITUKI2_10, HB_C_KOROKOPO_12, HB_C_KOROKIPO_3, WSERCsf_G4, HCNKIsf_G7, HCNTHsf_F8, HPRMAwf_D10 or RM11-1a.

Current application further provides the use of any of the yeast strains of the third aspect and its embodiments, for increasing the production of acetic acid in a yeast fermentation. “Acetic acid” (systematically named ethanoic acid) as used herein refers the colorless liquid organic compound with the chemical formula CH₃ COOH (also written as CH₃CO₂H or C₂H₄O₂). Acetic acid is the second simplest carboxylic acid (after formic acid). It consists of a methyl group attached to a carboxyl group. It is an important chemical reagent and industrial chemical, used primarily in the production of cellulose acetic acid for photographic film, polyvinyl acetic acid for wood glue, and synthetic fibres and fabrics. In households, diluted acetic acid is often used in descaling agents. In the food industry, acetic acid is controlled by the food additive code E260 as an acidity regulator and as a condiment. As a food additive it is approved for usage in many countries. Acetic acid is also known as an antibiotic compound (e.g. Rhee et al 2003 Appl Environ Microbiol 69: 2959-2963; Ryssel et al 2009 Burns 35: 695-700; Fraise et al 2013 J Hosp Infec 84: 329-331).

In a further embodiment also methods of statistically significantly increasing the production of acetic acid in a yeast fermentation are provided. These methods comprise the step of adding any of the yeast strains of the third aspect and its embodiments to a fermentation medium. The methods optionally further comprise the step of measuring the level of acetic acid.

“Increasing” as used here refers to statistically significantly increasing the production of acetic acid compared to the production of acetic acid by a control or isogenic yeast strain comprising a wild-type or functional version of ALD6. An example of such wild-type or functional ALD6 allele is provided in SEQ ID No. 7, encoding a wild-type or functional Ald6 protein as depicted in SEQ ID No. 9.

The application also provides the use of a genetic inhibitor of ALD6 to develop a yeast strain with statistically significantly increased production of acetic acid. Said increased production is compared to a control or an isogenic yeast strain not comprising or not treated with the genetic inhibitor. In one embodiment, said genetic inhibitor is an inhibitor of the RNA interference technology or antisense technology or alternatively phrased is an RNA-silencing agent. Non-limiting examples of RNA-silencing agents are siRNA, shRNA, dsRNA, divalent siRNA (di-siRNA), antisense oligonucleotides (ASO), gapmer, microRNA, ribozyme, DNAzyme, nucleic acid aptamer, locked nucleic acid (LNA), bridged nucleic acid (BNA), ethyl bridged nucleic acid (ENA), peptide nucleic acid (PNA) or a morpholino oligonucleotide.

In another embodiment, said genetic inhibitor is a nuclease, more particularly a CRISPR-Cas, a TALEN, a meganuclease or a Zinc-finger nuclease. The application thus also provides a chimeric gene construct comprising a promoter active in yeast operably linked to a Crispr guide RNA targeting a ALD6 allele. A non-limiting example of said Crispr guide RNA targeting ALD6 is provided in the application, however given the state of the art of the Crispr technology and the available ALD6 sequence the skilled person would have no problem in selecting alternative guide RNAs for efficiently inhibiting the Ald6 function. In one particular embodiment, a yeast strain or an industrial yeast strain is provided comprising a chimeric gene construct comprising a promoter active said yeast strain operably linked to a Crispr guide RNA targeting ALD6 or targeting an ALD6 allele, wherein the expression of ALD6 in said yeast is statistically significantly reduced compared to that in a control yeast not comprising said chimeric gene construct. This thus means that the chimeric gene construct is responsible for the reduced expression of ALD6.

In yet another embodiment, the use of a genetic inhibitor of ALD6 to develop a yeast strain with statistically significantly increased production of acetic acid is provided wherein said genetic inhibitor is a disrupted, partially deleted or completely deleted ALD6 allele. More particularly, the ALD6 allele encoding SEQ ID No. 10 or depicted in SEQ ID No. 8. Said increased production is compared to a control yeast strain comprising a functional or wild-type ALD6 allele.

S. cerevisiae var. boulardii Strains with Increased Acetic Acid Production

In Example 4 of current application it is demonstrated that the capacity of acetic acid production by S. cerevisiae var. boulardii can be increased by disrupting, partially deleting or completely deleting the ALD6 alleles. S. cerevisiae var. boulardii is a known probiotic whose probiotic effect is believed to be at least partly due to the production of acetic acid, which can affect the growth of other microbes in the gastrointestinal tract (Offei et al 2019 Genome Res 29). Probiotics are defined as live microorganisms that confer beneficial effects on their hosts when administered in drug-like quantities. S. cerevisiae var. boulardii is the only yeast strain that is prescribed as probiotic against gastrointestinal diseases and it is commercially available from pharmacies worldwide. There are clinical trials supporting its application against Antibiotic Associated Diarrhoea (AAD) (Kotowska et al 2005 Aliment Pharmacol Ther 21; Duman et al. 2005 Eur J Gastroenterol Hepatol 17), gut inflammatory manifestations in HIV-1 patients (Villar-Garcia et al. 2015 J Acquir Immune Defic Syndr 68) and recurrent Clostridium difficile infections when combined with classic antibiotics (McFarland et al. 1994 JAMA 271). S. cerevisiae var. boulardii is also known to ameliorate diarrhoea as a result of gastrointestinal infections caused by enteropathogens such as Vibrio cholera, Enterohaemorrhagic E. coli (EHEC) and Enteropathogenic E. coli (EPEC) (Czerucka et al. 2007 Aliment Pharmacol Ther 26). Although previously considered as a different species, modern molecular phylogenetic methods tend to consider it as a variety of the baker's yeast, Saccharomyces cerevisiae (Mitterdorfer et al. 2002 J Appl Microbiol 93; Mackenzie et al. 2008 Yeast 25; van der Aa Kuhle and Jespersen 2003 Syst Appl Microbiol 26; Edwards-Ingram et al. 2004 Genome Res 14). Whole-genome sequencing has indeed revealed that S. cerevisiae var. boulardii shares a highly similar genomic content and sequence to S. cerevisiae (Khatri et al. 2013 Gut Pathog 5).

Therefore, in a fourth aspect, the application provides a S. cerevisiae var boulardii yeast strain comprising a disrupted, partially deleted or completely deleted ALD6 allele for use as a medicament or more particularly for use in the treatment or prevention of gastrointestinal disorders, even more particularly for use in the treatment or prevention of diarrhea, for use in reducing gastrointestinal discomfort, increasing gastrointestinal comfort, improving immune health and/or relieving constipation.

S. cerevisiae var. boulardii is a diploid yeast, thus in one embodiment the S. cerevisiae var. boulardii strain provided herein comprises a disrupted, partially deleted or completely deleted ALD6 allele in a homozygous or hemizygous form. Alternatively phrased, said S. cerevisiae var boulardii strain comprises a homozygous or hemizygous ALD6 allele compromising, partially abolishing or completely abolishing Ald6 function. The application also provides a haploid segregant of said S. cerevisiae var. boulardii strain. In particular embodiments, the ALD6 alleles has been disrupted or deleted by homologous recombination. Many S. cerevisiae var. boulardii strains are available including several strains that are commercially available.

In a particular embodiment, at least one of both disrupted, partially deleted or completely deleted ALD6 alleles comprises an A to C mutation on position 184 of SEQ ID No. 7 or encodes an Ald6 protein comprising a T to P mutation on position 62 of SEQ ID No 9 or encodes the Ald6 protein as depicted in SEQ ID No. 10 or is the ALD6 allele as depicted in SEQ ID No. 8.

In another embodiment, a beverage, food supplement, food or feed product, dietary supplement or pharmaceutical composition comprising the S. cerevisiae var. boulardii strain of the fourth aspect and its embodiments is provided. The term “food or feed product” is intended to encompass any consumable matter of either plant or animal origin or of synthetic sources that contain a body of nutrients such as a carbohydrate, protein, fat vitamin, mineral, etc. The product is intended for the consumption by humans or by animals, such as domesticated animals, for example cattle, horses, pigs, sheep, goats, and the like. Pets such as dogs, cats, rabbits, guinea pigs, mice, rats, birds (for example chickens or parrots), reptiles and fish (for example salmon, tilapia or goldfish) and crustaceans (for example shrimp). The food product may be liquid or solid. It may include but is not limited to a liquid fermented solution such as milk or yoghurt. The feed product may include but is not limited to pelleted feeds or pet feed for example a snack bar, crunchy treat, cereal bar, snack, biscuit, pet chew, pet food, and pelleted or flaked feed for aquatic animals.

The application also provides methods of treating or preventing gastrointestinal disorders, more particularly diarrhea in a human or animal or of maintaining or improving the health of the gastrointestinal tract in a human or animal, said method comprising administering to said human or animal a dietary supplement or pharmaceutical composition, wherein said dietary supplement or pharmaceutical composition comprises a S. cerevisiae var boulardii strain comprising a homozygously or hemizygously disrupted, partially deleted or completely deleted ALD6. In a particular embodiment, at least one of both disrupted, partially deleted or completely deleted ALD6 alleles comprises an A to C mutation on position 184 of SEQ ID No. 7 or encodes an Ald6 protein comprising a T to P mutation on position 62 of SEQ ID No 9 or encodes the Ald6 protein as depicted in SEQ ID No. 10 or is the ALD6 allele as depicted in SEQ ID No. 8.

In other embodiments, said maintaining or improving the health of the gastrointestinal tract comprises reducing the number of pathogenic bacteria found in the faeces of said human or animal. In more particular embodiments, said pathogenic bacteria are selected from the group consisting of Clostridia, Escherichia, Salmonella, Shigella and mixtures thereof. The herein provided S. cerevisiae var boulardii must arrive in large numbers in the gut in order to settle there, and must not be destroyed by stomach acid as it passes the stomach. Therefore, in particular embodiments, said dietary supplement or pharmaceutical composition comprises a therapeutically effective amount of said S. cerevisiae var boulardii yeast strains. In more particular embodiments, said therapeutically effective amount is an amount of more than 10⁶ CFU (colony forming units), or of more than 10⁷ CFU, or of more than 10⁸ CFU or of more than 10⁹ CFU of said yeast per gram or per ml of said supplement or composition, or comprises between 10⁵ and 10¹⁵ CFU, or between 10⁶ and 10¹² CFU, or between 10⁷ and 10¹¹ CFU, or between 10⁸ and 6×10¹⁰ CFU, or between 10⁹ and 2×10¹⁰ CFU of said yeast per gram or per ml of said supplement or composition.

The above disclosed methods are both for treating and preventing gastrointestinal disorders. Indeed, administration of certain live probiotic yeasts can help restore optimal intestinal flora in animals such as cattle, especially after stressful situations such as transport to a feedlot (Gedek, B., “Probiotics in Animal Feeding—Effects on Performance and Animal Health,” Feed Magazine, November 1987) but regular administration of probiotics also increase nutrient absorption efficiency and help control the proliferation of harmful microorganisms in the animals' digestive tracts that could otherwise cause disease conditions adversely affecting rates of animal development and weight gain.

### An URK1 Allele Providing Increased Production of Isobutanol

In a fifth aspect, a yeast strain is provided comprising a disrupted, partially deleted or completely deleted URK1 allele. This is equivalent as saying that a yeast strain is provided comprising a deficient URK1 allele.

URK1 (YNR012W; SGD:S000005295) encodes a uridine/cytidine kinase and is a component of the pyrimidine ribonucleotide salvage pathway that converts uridine into UMP and cytidine into CMP. The wild-type DNA sequence is depicted in SEQ ID No. 5.

In one embodiment, the yeast strain comprising a disrupted, partially deleted or completely deleted URK1 allele is a haploid yeast strain or a haploid segregant from a diploid yeast. In another embodiment, said yeast strain is a diploid yeast strain. In yet another embodiment, said yeast strain is a diploid yeast strain and the URK1 allele is present in homozygous form, meaning that both URK1 alleles of said diploid yeast strain are a disrupted, partially deleted or completely deleted URK1 allele. In a further embodiment, both URK1 alleles are identical. In another further embodiment, both URK1 alleles are different. In another embodiment, a yeast strain is provided comprising a homozygous or hemizygous mutant UKR1 allele wherein said URK1 allele compromises, partially abolishes or completely abolishes Urk1 function.

In another particular embodiment, said yeast strain of the fifth aspect and any of its embodiments is an industrial yeast strain. In another particular embodiment, said yeast strain of the fifth aspect and any of its embodiments is a Saccharomyces cerevisiae strain. In yet another particular embodiment, said yeast strain or said S. cerevisiae strain is an engineered or recombinant yeast or S. cerevisiae strain.

Current application further provides the use of any of the yeast strains of the fifth aspect and its embodiments, for increasing the production of isobutanol in a yeast fermentation. In a further embodiment also methods of statistically significantly increasing the production of isobutanol in a yeast fermentation are provided. These methods comprise the step of adding any of the yeast strains of the fifth aspect and its embodiments to a fermentation medium. The methods optionally further comprise the step of measuring the level of isobutanol. “Increasing” as used here refers to statistically significantly increasing the production of isobutanol compared to the production of isobutanol by a control or isogenic yeast strain comprising a wild-type or functional version of URK1. An example of such wild-type or functional URK1 allele is provided in SEQ ID No. 5.

The application also provides the use of a genetic inhibitor of URK1 to develop a yeast strain with statistically significantly increased production of isobutanol. Said increased production is compared to a control or an isogenic yeast strain not comprising or not treated with the genetic inhibitor. In one embodiment, said genetic inhibitor is an inhibitor of the RNA interference technology or antisense technology or alternatively phrased is an RNA-silencing agent. Non-limiting examples of RNA-silencing agents are siRNA, shRNA, dsRNA, divalent siRNA (di-siRNA), antisense oligonucleotides (ASO), gapmer, microRNA, ribozyme, DNAzyme, nucleic acid aptamer, locked nucleic acid (LNA), bridged nucleic acid (BNA), ethyl bridged nucleic acid (ENA), peptide nucleic acid (PNA) or a morpholino oligonucleotide.

In another embodiment, said genetic inhibitor is a nuclease, more particularly a CRISPR-Cas, a TALEN, a meganuclease or a Zinc-finger nuclease. The application thus also provides a chimeric gene construct comprising a promoter active in yeast operably linked to a Crispr guide RNA targeting a URK1 allele. A non-limiting example of said Crispr guide RNA targeting URK1 is provided in the application, however given the state of the art of the Crispr technology and the available URK1 sequence the skilled person would have no problem in selecting alternative guide RNAs for efficiently inhibiting the Urk1 function. In one particular embodiment, a yeast strain or an industrial yeast strain is provided comprising a chimeric gene construct comprising a promoter active said yeast strain operably linked to a Crispr guide RNA targeting URK1 or targeting an URK1 allele, wherein the expression of URK1 in said yeast is statistically significantly reduced compared to that in a control yeast not comprising said chimeric gene construct. This thus means that the chimeric gene construct is responsible for the reduced expression of URK1.

In yet another embodiment, the use of a genetic inhibitor of URK1 to develop a yeast strain with statistically significantly increased production of isobutanol is provided, wherein said genetic inhibitor is a disrupted, partially deleted or completely deleted URK1 allele. Said increased production is compared to a control yeast strain comprising a functional or wild-type URK1 allele.

### Terminology as Used in Describing the Aspects of the Invention

“A yeast strain comprising a disrupted, partially deleted or completely deleted allele” as used herein refers to a yeast strain having disrupted, partially deleted or completely deleted functional expression of said allele or having disrupted, partially deleted or completely deleted function of said allele.

Means and methods to disrupt, partially delete or completely delete a gene, an allele or protein are well known in the art. The skilled person can select from a plethora of techniques to affect the expression or function of Whi2. One very efficient technique is the Crispr/Cas technology which has also been used in the Examples of this application. At the DNA level, disruption, partial deletion or complete deletion can for example be achieved by removing or disrupting a gene or by mutations in the promoter of the gene. Non-limiting examples are knock-outs or loss-of-function mutations but also gain-of-function mutations and dominant negative mutations can disrupt the functional expression or inhibit the formation of a functional mRNA molecule. A “knock-out” can be a gene knockdown (leading to reduced gene expression) or the gene can be knocked out by a mutation such as, a point mutation, an insertion, a deletion, a frameshift, or a missense mutation by techniques known in the art. The lack of transcription can e.g. be caused by epigenetic changes (e.g. DNA methylation) or by loss-of-function mutations. A “loss-of-function” or “LOF” mutation as used herein is a mutation that prevents, reduces or abolishes the function of a gene product as opposed to a gain-of-function mutation that confers enhanced or new activity on a protein. Both dominant negative or LOF mutations can be caused by a wide range of mutation types, including, but not limited to, a deletion of the entire gene or part of the gene, splice site mutations, frame-shift mutations caused by small insertions and deletions, nonsense mutations, missense mutations replacing an essential amino acid and mutations preventing correct cellular localization of the product.

Disruption of an allele can thus be achieved by inserting a DNA fragment in the base sequence of said allele or deleting a portion of said allele so that the allele cannot function any longer. As a result of gene (or allele) disruption, the gene (or allele) cannot be transcribed into mRNA, hence the structural gene is not translated, or the transcription product mRNA becomes incomplete, hence mutation or deletion occurs in the amino acid sequence of the translation product structural protein, rendering the protein incapable of performing the original function. In order to disrupt the ALD6, SUC2, UKR1 or IMA1 allele herein described, any site may be disrupted, for example, a promoter site of the alleles, an open reading frame (ORF) site, and a terminator site, or combination thereof may be disrupted. Gene disruption can also be carried out by deleting the whole ALD6, SUC2, UKR1 or IMA gene. Therefore in alternative embodiments, the yeast strains herein provided comprise a completely deleted ALD6, SUC2, UKR1 or IMA1 allele or a yeast strain devoid of the ALD6, SUC2, UKR1 or IMA1 allele or deficient of the ALD6, SUC2, UKR1 or IMA1 allele.

The alleles herein disclosed can be disrupted, for example, by transforming a plasmid or a fragment thereof for disrupting the alleles into yeast, and causing homologous recombination of the DNA fragment contained in the transformed plasmid or fragment thereof with the gene on yeast genome. In case that a plasmid for disruption of a gene or a fragment thereof and the gene on the yeast genome have a homology to an extent for causing homologous recombination, homologous recombination is caused.

It will be understood that methods for gene disruption in yeast and other microorganisms are well known, and the particular method used to reduce or abolish the expression of the endogenous gene is not critical to the invention. In one embodiment, disruption can be accomplished by homologous recombination, whereby the gene to be disrupted is interrupted (e.g. by the insertion of a selectable marker gene) or made inoperative (e.g. “gene knockout”). Methods for gene knockout and multiple gene knockout are well known. See e.g. Rothstein 2004, “Targeting, Disruption, Replacement, and Allele Rescue: Integrative DNA Transformation in Yeast” In: Guthrie et al., Eds. Guide to Yeast Genetics and Molecular and Cell Biology, Part A, p. 281-301; Wach et al., 1994, “New heterologous modules for classical or PCR-based gene disruptions in Saccharomyces cerevisiae” Yeast 10:1793-1808. Methods for insertional mutagenesis are also well known. See e.g. Amberg et al., eds., 2005, Methods in Yeast Genetics, p. 95-100; Fickers et al., 2003, “New disruption cassettes for rapid gene disruption and marker rescue in the yeast Yarrowia lipolytica” Journal of Microbiological Methods 55:727-737; Akada et al., 2006, “PCR-mediated seamless gene deletion and marker recycling in Saccharomyces cerevisiae” Yeast 23:399-405; Fonzi et al., 1993, “Isogenic strain construction and gene mapping in Candida albicans” Genetics 134:717-728. Other methods to disrupt a gene in a microorganism include the use of nucleases, such as zinc-finger nucleases (ZFNs), Transcription Activator-Like Effector Nucleases (TALENs), meganucleases but especially the CRISPR-Cas system.

“Nucleases” as used herein are enzymes that cut nucleotide sequences. These nucleotide sequences can be DNA or RNA. If the nuclease cleaves DNA, the nuclease is also called a DNase. If the nuclease cuts RNA, the nuclease is also called an RNase. Upon cleavage of a DNA sequence by nuclease activity, the DNA repair system of the cell will be activated. Yet, in most cases the targeted DNA sequence will not be repaired as it originally was and small deletions, insertions or replacements of nucleic acids will occur, mostly resulting in a mutant DNA sequence. ZFN are artificial restriction enzymes generated by fusing a zinc finger DNA-binding domain to a DNA cleavage domain. Zinc finger domains can be engineered to target desired DNA sequences, which enables zinc-finger nucleases to target a unique sequence within a complex genome. By taking advantage of endogenous DNA repair machinery, these reagents can be used to precisely alter the genomes of simple and higher organisms. Other technologies for genome customization that can be used to knock out genes are meganucleases and TAL effector nucleases (TALENs, Cellectis bioresearch). A TALEN is composed of a TALE DNA binding domain for sequence-specific recognition fused to the catalytic domain of an endonuclease that introduces double strand breaks (DSB). The DNA binding domain of a TALEN is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). Another recent and very popular genome editing technology is the CRISPR-Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway and has been modified to edit basically any genome. By delivering the Cas nuclease (in many cases Cas9) complexed with a synthetic guide RNA (gRNA) in a cell, the cell's genome can be cut at a desired location depending on the sequence of the gRNA, allowing existing genes to be removed and/or new one added and/or more subtly removing, replacing or inserting single nucleotides (e.g. DiCarlo et al 2013 Nucl Acids Res doi:10.1093/nar/gkt135; Sander & Joung 2014 Nat Biotech 32:347-355). Therefore, yeast strains are also provided herein in which the ALD6, SUC2, UKR1 or IMA1 allele has been disrupted or deleted by using nuclease technology, more particularly by means of the CRISPR-Cas technology.

“Homozygous” refers to having identical alleles for a single trait. An “allele” represents one particular form of a gene. Alleles can exist in different forms and diploid organisms typically have two alleles for a given trait. A homozygous mutant SUC2, ALD6, URK1 or IMA1 allele thus means that all SUC2, ALD6, URK1 or IMA1 alleles are identical.

“Hemizygous” refers to having only one allele for a single trait or gene. In case of a diploid organism thus only one allele of its pairs is present, while all other genes are represented by two alleles. This can for example be achieved by deleting one allele of a gene or by introducing one allele of a gene that is not present in an organism.

A “mutation on nucleic acid position x” is equivalent as saying that the nucleobase on position x is mutated. With “mutation on nucleic acid position x” as used herein, it is thus meant that nucleobase x from the wild-type allele is mutated. “Position x” or “nucleobase x” as used herein refers to the nucleobase that is x-1 positions removed downstream from the first nucleobase (i.e. adenosine) from the start codon.

“Nucleobases” are nitrogen-containing biological compounds that form nucleosides, which in turn are components of nucleotides; all which are monomers that are the basic building blocks of nucleic acids. Often simply called bases, as in the field of genetics, the ability of nucleobases to form base-pairs and to stack one upon another leads directly to long-chain helical structures such as ribonucleic acid (RNA) and deoxyribonucleic acid (DNA). There are four so-called DNA-bases: adenine (A), cytosine (C), guanine (G) and thymine (T).

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g. peptide nucleic acids).

By “encoding” or “encodes” or “encoded”, with respect to a specified nucleic acid, is meant comprising the information for transcription into an RNA molecule and in some embodiments, translation into the specified protein or amino acid sequence. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

Yeasts are eukaryotic, single-celled microorganisms classified as members of the fungus kingdom and like all fungi, yeast may have asexual and sexual reproductive cycles. The most common mode of vegetative growth in yeast is asexual reproduction by budding. Here, a small bud or daughter cell, is formed on the parent cell. The nucleus of the parent cell splits into a daughter nucleus and migrates into the daughter cell. The bud continues to grow until it separates from the parent cell, forming a new cell. This reproduction cycle is independent of the yeast's ploidy, thus both haploid and diploid yeast cells can duplicate as described above. Haploid cells have in general a lower fitness and they often die under high-stress conditions such as nutrient starvation, while under the same conditions, diploid cells can undergo sporulation, entering sexual reproduction (meiosis) and producing a variety of haploid spores or haploid segregants, which can go on to mate (conjugate), reforming the diploid. Haploid cells contain one set of chromosomes, while diploid cells contain two. A haploid segregant as used herein is equivalent as a haploid spore, the result of sporulation.

The budding yeast Saccharomyces cerevisiae reproduces by mitosis as diploid cells when nutrients are abundant, but when starved, this yeast undergoes meiosis to form haploid spores. Haploid cells may then reproduce asexually by mitosis. Importantly, S. cerevisiae var. boulardii is sporulation deficient (Edwards-Ingram et al 2007 Appl Environ Microbiol 73: 2458-2467) and thus does not have the ability to naturally form haploid spores or haploid segregants.

“Engineering” or “engineered” as used herein refers to genetic engineering, a technique whereby an organism's genome is modified using biotechnology. This includes but is not limited to the transfer of genes within and across species boundaries, deleting fragments of genes or deleting whole genes, modifying the DNA sequence of an organism by deleting, inserting or substituting one or more nucleic acid molecules. Means and methods to engineer microorganisms, particularly yeasts are well known by the person skilled in the art. The most known techniques involve traditional genetic transformation of yeast and recombinant DNA techniques. Nowadays, the most attractive technique to engineer a microorganism is by the use of nucleases, such as zinc-finger nucleases (ZFNs), Transcription Activator-Like Effector Nucleases (TALENs), meganucleases but especially the CRISPR-Cas system as described earlier.

Alcohol by volume (abbreviated as ABV, abv, or alc/vol) is a standard measure of how much alcohol (i.e. ethanol) is contained in a given volume of an alcoholic beverage (expressed as a volume percent or vol %). It is defined as the number of milliliters (ml) of pure ethanol present in 100 ml of solution at 20° C. The number of milliliters of pure ethanol is the mass of the ethanol divided by its density at 20° C., which is 0.78924 g/ml. The ABV standard is used worldwide.

The term “endogenous” as used herein, refers to substances (e.g. genes or proteins) originating from within an organism, tissue, or cell. Analogously, “exogenous” is any material originated outside of an organism, tissue, or cell, but that is present (and typically can become active) in that organism, tissue, or cell.

A “promoter” is a DNA sequence comprising regulatory elements, which mediate the expression of a nucleic acid molecule. For expression, the nucleic acid molecule must be linked operably to or comprise a suitable promoter which expresses the gene at the right point in time and with the required spatial expression pattern. The term “operably linked” as used herein refers to a functional linkage between the promoter sequence and the gene of interest, such that the promoter sequence is able to initiate transcription of the gene of interest or for example of a Crispr guide RNA. A promoter that enables the initiation of gene transcription in a eukaryotic cell is referred to as being “active”. To identify a promoter which is active in a eukaryotic cell, the promoter can be operably linked to a reporter gene after which the expression level and pattern of the reporter gene can be assayed. Suitable well-known reporter genes include for example beta-glucuronidase, beta-galactosidase or any fluorescent or luminescent protein. The promoter activity is assayed by measuring the enzymatic activity of the beta-glucuronidase or beta-galactosidase. Alternatively, promoter strength may also be assayed by quantifying mRNA levels or by comparing mRNA levels of the nucleic acid, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994).

A “chimeric gene” or “chimeric construct” is a recombinant nucleic acid sequence in which a promoter or regulatory nucleic acid sequence is operably linked to, or associated with, a nucleic acid sequence that codes for a mRNA (optionally further encoding an amino acid sequence), such that the regulatory nucleic acid sequence is able to regulate transcription or expression of the associated nucleic acid coding sequence. Importantly, in a chimeric gene construct as used herein the regulatory nucleic acid sequence of the chimeric gene is not operably linked to the associated nucleic acid sequence as found in nature.

The term “a 3′ end region involved in transcription termination or polyadenylation” encompasses a control sequence which is a DNA sequence at the end of a transcriptional unit which signals 3′ processing or polyadenylation of a primary transcript and is involved in termination of transcription.

The control sequence for transcription termination or terminator can be derived from a natural gene or from a variety of genes. For expression in yeast the terminator to be added may be derived from, for example, the TEF or CYC1 genes or alternatively from another yeast gene or less preferably from any other eukaryotic or viral gene.

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The terms or definitions provided herein are solely to aid in the understanding of the invention. Unless specifically defined herein, all terms used herein have the same meaning as they would to one skilled in the art of the present invention. Practitioners are particularly directed to Michael R. Green and Joseph Sambrook, Molecular Cloning: A Laboratory Manual, 4th ed., Cold Spring Harbor Laboratory Press, Plainsview, New York (2012); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art. It is to be understood that although particular embodiments, specific configurations as well as materials and/or molecules, have been discussed herein for cells and methods according to the present invention, various changes or modifications in form and detail may be made without departing from the scope and spirit of this invention. The Examples described below are provided to better illustrate particular embodiments, and they should not be considered limiting the application. The application is limited only by the claims.

Additional to the above detailed description of the invention, the following experimental details further enable the skilled person to put all details of the invention into practice.

### EXAMPLES

### Example 1. Identification of QTLs that Affect Industrially-Relevant Phenotypes

1125 F6 segregants were obtained from a cross of two phenotypically divergent S. cerevisiae strains, RM11-1a, a natural vineyard isolate, and YJM975α, originally isolated from an immunocompromised patient in Italy (She & Jarosz, 2018). For each of the 1125 segregants and the two parental yeasts, a fermentation reaction in medium and conditions mimicking industrial wort fermentation was set up. For each of these 1125 fermentations, the inventors of the application measured 18 different industrially-relevant parameters, including the production of primary and secondary metabolites. In addition, all 1125 segregants were screened for their resistance to various stress factors and consumption of maltose. This large set of phenotypic data was subsequently combined with the available genome sequences of each of the segregants and analyzed using the pipeline developed by She & Jarosz (2018) to identify QTLs underlying the phenotypes (see methods).

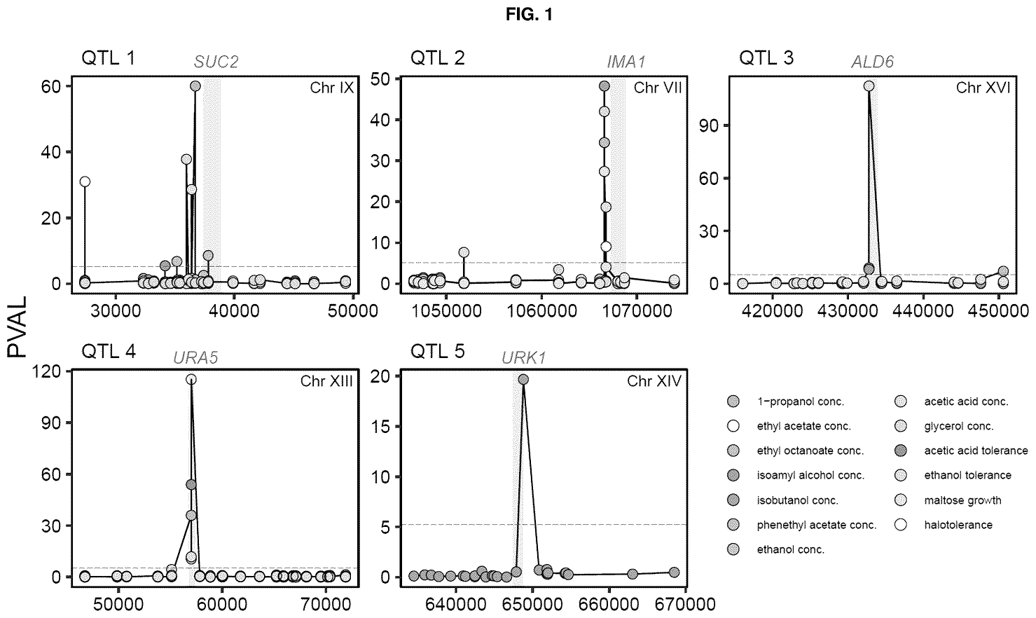

We selected five QTLs that are predicted to affect multiple industrially-relevant phenotypes for further investigation (FIG. 1). The QTLs were selected because they are linked to a broad range of different traits, from the production of primary metabolites such as ethanol, glycerol and acetic acid to secondary metabolites including valuable compounds like 1-propanol, ethyl acetate and isobutanol, as well as tolerance towards salt (FIG. 1). For each of these QTLs, we followed a similar strategy to identify the causative alleles or mutation and their respective phenotypic effects. In a first step, we tested the contribution of several candidate genes located within the predicted QTLs to a given phenotype by checking the phenotypic effect of deleting the gene in both parental genetic backgrounds. Apart from their central location in the predicted QTL regions, the target genes were selected either because they contain non-synonymous variants between the parent strains RM11-1a and YJM975α and/or have a molecular phenotype (enzymatic activity or transcriptional activity) that could be linked to the specific phenotype under investigation.

### Example 2. Variation in SUC2 Causes Differences in the Production of Alcohol and Various Other Metabolites

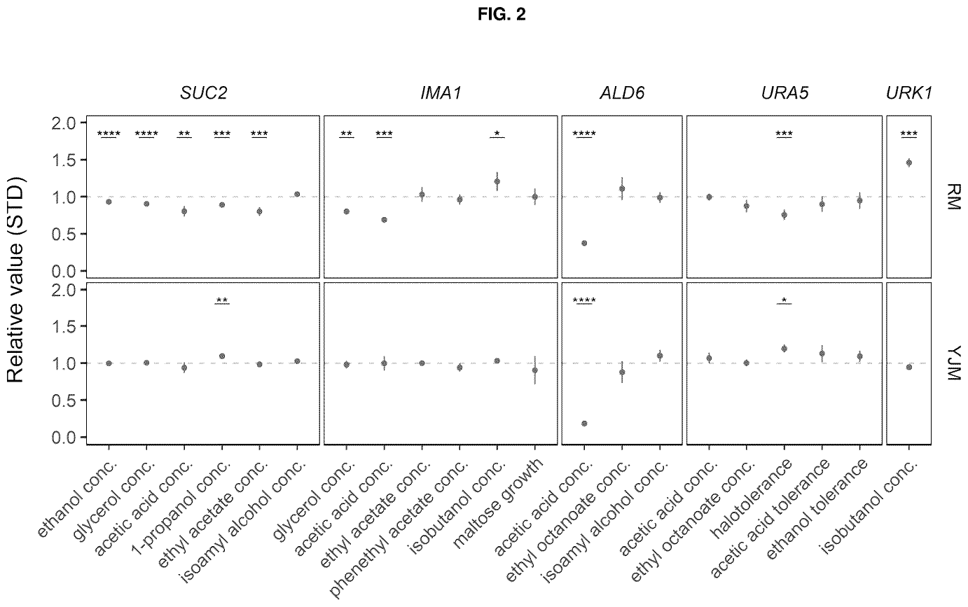

SUC2 was one of the candidate alleles within QTL1. Deletion of SUC2 in strain RM11-1a led to 6.5% reduction (p<=0.0001) in the final ethanol concentration in the sample, while no effect was observed in strain YJM975α (FIG. 2). Similarly, SUC2 deletion also led to reduced formation of 1-propanol (−10.8%; p<=0.001), ethyl acetate (−17.9%; p<=0.001), acetic acid (−16.5%; p<=0.01), and glycerol (−10.8%; p<=0.0001) in RM11-1a, but had no effect in the strain YJM975α, except for the formation of 1-propanol (+8.6%; p<=0.01) (FIG. 2).

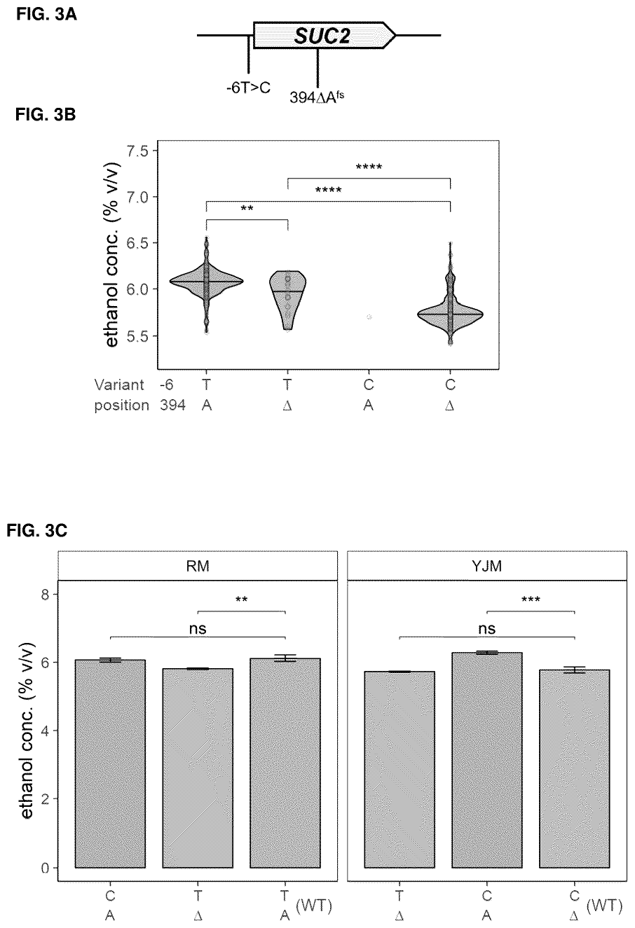

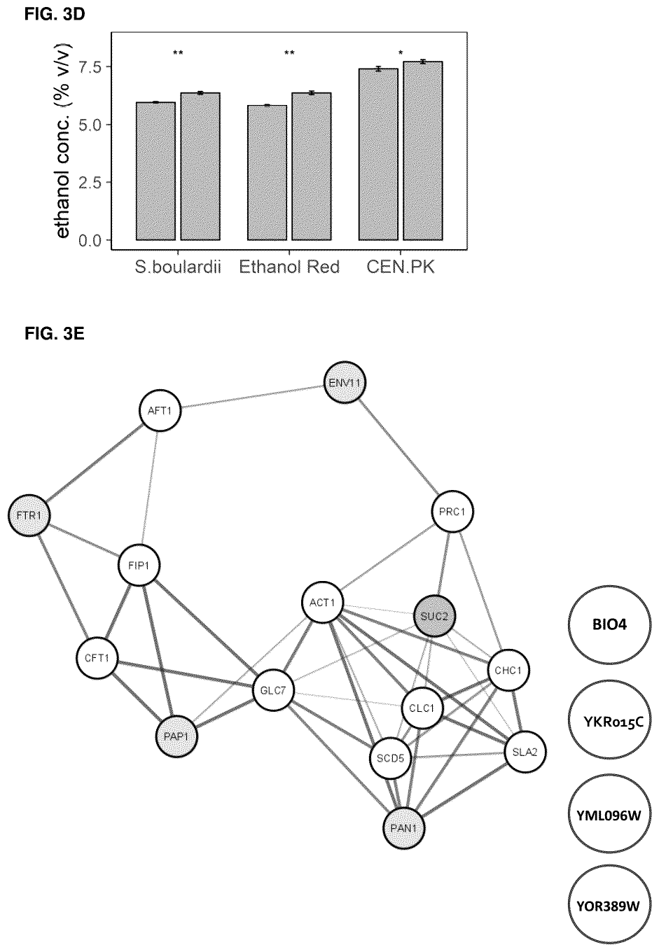

Next, we attempted to identify the exact causative genetic variation in SUC2 that is responsible for the observed phenotypes. Our QTL pipeline highlighted one frameshift variant in the ORF of the SUC2 gene (SUC23944) with high significance (PVAL >5.2) from several traits. However, since this variant could not be unambiguously distinguished from a nearby variant located at position-6 in the promoter of SUC2, we also included this variant for our validation (FIG. 3A). Segregants inheriting the entire haplotype block from RM11-1a produced higher amounts of ethanol (FIG. 3B). Using the CRISPR-Cas toolbox, we swapped the frameshift variant (SUC23944) between the wild type parent strains. This reverted the respective phenotypes compared to the respective wild-type strain, confirming that this frameshift mutation is at the basis of the QTL (FIG. 3C). This variant locates immediately at 5′ of the known catalytic site of Suc2 (Mohandesi et al. 2017), which seems to alter the affinity of the enzyme, as residual sucrose was detected in the finished fermentation sample (data not shown). Moreover, swapping of this variant between the founder strains led to opposing effect on the formation of ethanol, acetic acid, glycerol and 1-propanol (FIG. 4), confirming the frameshift variant is the main determinant located near the pleotropic SUC2 QTL.

To test if the truncated SUC2 allele leads to reduced ethanol production in other strains containing an intact SUC2 allele, we introduced the SUC23944 frameshift variant into a probiotic diploid strain Saccharomyces cerevisiae var. boulardii, an industrial bioethanol diploid strain Ethanol Red, and a haploid laboratory chassis strain CEN.PK. Hemizygous or homozygous introduction of this frameshift variant (in haploid or diploid strains respectively) indeed led to reduced ethanol formation in all three strains (FIG. 3D), substantiating the effect of a truncated SUC2 allele on ethanol formation. Next, we investigated whether other polymorphisms located in genes that interact with SUC2 also show a detectable effect on ethanol production (FIG. 3E). However, we did not observe a clear relationship between these individual polymorphisms and ethanol production.

### Example 3. Incomplete IMA1 Locus Impacts Glycerol, Acetic Acid and Isobutanol Production

Within QTL2, linked to several traits such as glycerol and acetic acid, several polymorphisms were identified in the intergenic region between BIO2 and IMA1 genes (approximately 2.2 kb) that are located within the subtelomeric region of chromosome VII (FIG. 1). While deletion of BIO2 in either of the strain RM11-1a and YJM975α did not affect any of the phenotypes to which the QTL was linked (data not shown), the deletion of IMA1 in RM11-1a led to reduced production of glycerol (−21.7%; p<=0.01) and acetic acid (−30.8%; p<=0.001), and increased production of isobutanol. By contrast, IMA1 deletion in strain YJM975α did not result in any significant changes (FIG. 2).

The IMA1 gene encodes for the major isomaltase required for isomaltose utilization, which also exhibits alpha-1,2 glucosidase activity on sucrose and kojibiose (Teste et al., 2010; Voordeckers et al., 2012). Near the IMA1 locus, one intergenic variant (IMA1^{+659G>C}) was predicted to influence several traits with strong significance (PVAL >50). Comparison of the IMA1 locus between strain RM11-1a and YJM975α revealed one missense mutation (IMA1^{1007A>T}), which has been reported to have a deleterious effect to the growth on raffinose, sucrose and maltose (Jackobson et al., 2019). Yet, swapping either of the two variants between the two strains did not lead to any significant change in the phenotypes that were observed in the ima1 deletion mutant (data not shown). However, a closer evaluation of the YJM975α genome sequence revealed that it lacks approximately 8 kb of the genomic region directly upstream of IMA1, including part of the 5′end of IMA1 (207 bp) as well as the entire coding regions of MAL13, encoding the activator protein that activates the permease and hydrolase when substrate is present, and MAL11, encoding the maltose/isomaltose permease (Novak et al., 2004). This 8 kb deletion segregates in the F6 progeny and correlates with the growth on maltose, with segregants containing the intact MAL-IMA1 locus showing more efficient growth on maltose, indicating that this structural variation is at the heart of this QTL.

### Example 4. Variation in ALD6 Drives Changes in Acetic Acid Production

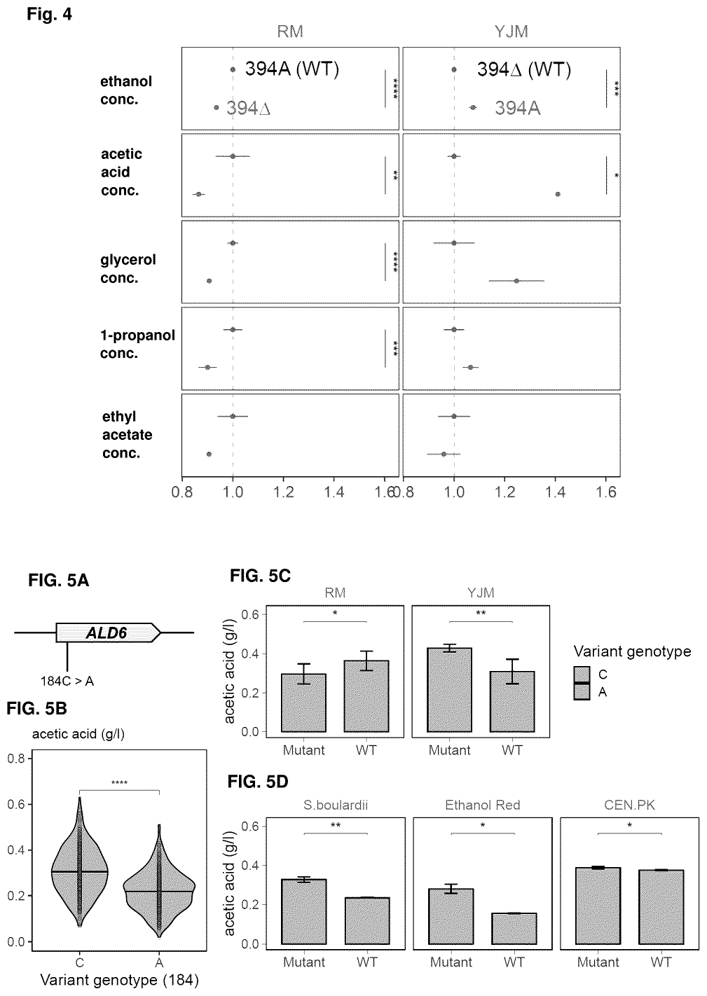

For QTL3, deletion of ALD6 resulted in significantly reduced acetic acid production in both parental strains, by 63.2% in RM11-1a (p<=0.0001) and by 81.7% (p<=0.0001) in YJM975α (FIG. 2). ALD6 encodes cytosolic aldehyde dehydrogenase, an enzyme required for the conversion of acetaldehyde to acetate. Our QTL pipeline linked the changes in acetic acid production to a missense variant (ALD6^{184A>C}) located in the ALD6 ORF (FIG. 5A). Segregants inheriting the haplotype from RM11-1a (ALD6^{184C}) produced higher amounts of acetic acid compared to the ones containing the YJM975α allele (ALD6^{184A}) (FIG. 5B). Swapping the specific alleles between RM11-1a and YJM975α led to a 22.3% (p<=0.05) reduction in acetic acid formation in RM11-1a, and a 22.0% (p<=0.001) increase in YJM975α, confirming that this variation is indeed the driver in this QTL (FIG. 5C).

To test if the alternative ALD6^{184C} variant could increase acetic acid in other strains containing the ALD6^{184A} variant, we replaced the ALD6^{184A} variant in strain Ethanol Red and strain CEN.PK as well as in strain S. cerevisiae var boulardii, whose probiotic effect is believed to be at least partly due to the production of acetic acid, which can affect the growth of other microbes in the gastrointestinal tract (Offei et al., 2019). Replacement of the threonine residue by the proline residue indeed led to increased acetic acid formation in all three strains (FIG. 5D).

### Example 5. URA5 is Involved in Salt Tolerance

Deletion of URA5 led to salt sensitivity in strain RM11-1a, while improving salt tolerance in strain YJM975α (FIG. 2), suggesting that URA5 is the causal element in QTL4 for the differential halotolerance between the two strains. Further analysis identified a missense variant within URA5 (URA5^{266G>T}), which encodes for the major orotate phosphoribosyl transferase (OPRTase), catalyzing the fifth enzymatic step in de novo biosynthesis of pyrimidines (Umezu et al., 1971).

### Example 6. URK1 Modulates Isobutanol Production

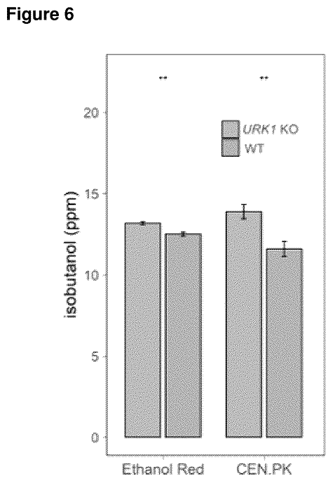

For QTL5, deletion of URK1 resulted in significantly increased production of isobutanol by 47.2% in RM11-1a (FIG. 2). URK1 encodes pyrimidine kinase, an enzyme involved in the deoxyribonucleotide salvage pathway. Interestingly, the deletion of URK1 in Ethanol Red and CEN.PK also led to increased production of isobutanol (FIG. 6).

### Materials and Methods

### Yeast Strains

The yeast strains were routinely maintained on solid YPD medium containing 10 g L-1 yeast extract, 20 g L-1 peptone, 20 g L-1 glucose, and 15 g L-1 agar. Frozen stocks of all strains were maintained at −80° C. using a glycerol-based storage medium (20 g L-1 Bacto peptone, 10 g L-1 yeast extract, 20 g L-1 glucose, 250 mL L-1 glycerol).

### General Molecular Biology and Microbiological Techniques

Genomic DNA extraction from yeast was performed using Phenol-Chloroform-Isoamyl alcohol (PCI) according to the method described by Hoffman & Winston (1987). Plasmids were isolated from E. coli DH5α cells from overnight cultures in lysogeny broth (LB) containing 10 g L-1 peptone, 5 g L-1 yeast extract and 10 g L-1 NaCl (pH 7.0) with 100 mg L-1 carbinicilin by using the Qiagen Miniprep Kit (Qiagen, Germany). Transformation of yeast cells with plasmids as well as PCR-amplified DNA fragments for genomic integration was performed using LiAc/PEG method described by Gietz et al. (1995).

### Lab-Scale Fermentation in Wort

Yeast pre-culture was inoculated overnight at 20° C. in test-tubes containing 3 mL of 10 g L-1 yeast extract, 20 g L-1 peptone, 40 g L-1 maltose medium (YPMal). After 16 h of incubation, 1 mL of the pre-culture was used to inoculate 50 mL of the YPMal medium in 250-ml Erlenmeyer flask and propagated in the same conditions as the pre-culture for 3 days. Notably, 10% of the segregants showed growth defects during pre-culturing and thus were exempted from further fermentation. The propagated cells were then used for inoculation of the fermentation medium, i.e. 160 Plato (160P) wort prepared by in-house brewery at a pitching rate of 106 cells mL-1. A blank wort medium were included in each batch of fermentation. Fermentations were performed in 250-mL Schott bottles with a water lock placed on each bottle and stirred at 150 rpm for 7 days at 20° C. Weight loss was monitored daily to follow the progress of fermentation. After 7 days, the fermentations were terminated on ice to minimize evaporation of volatile compounds and sampled for analytical analysis. Fermentation of all segregants was individually performed only once.

### Analytical Methods

Quantification of yeast aroma production was carried out using headspace gas chromatography coupled with flame ionization detection (HS-GC-FID; Shimadzu, Japan). The GC was calibrated with 8 important aroma compounds, including isobutanol, isoamyl alcohol, ethyl acetate, ethyl hexanoate, ethyl octanoatem isoamyl acetate and phenetyl acetate, using 2-heptanol as the internal standard. Specifications of the GC system and the sample preparation are as described in Steensels et al. (2014). Ethanol measurements were performed with the Alcolyzer Beer DMA 4500M (Anton Paar, Austria). Filtered samples (0.15 mm paper filter) were measured for the level of glycerol, acetic acid and sulfite using Thermo Scientific Gallery discrete photometric analyzer (Thermo Fisher Scientific, USA). Sugar concentrations were determined by Dionex Liquid Chromatography (Thermo Fisher Scientific, USA), which was calibrated for maltose, sucrose, glucose and fructose using raffinose as the internal standard.

### Yeast Phenotyping

F6 segregants taken from frozen stock were pinned on solid YPD medium using a Singer ROTOR robotic pinning instrument, with which cells were subsequently transferred to various solid media (YPD with stress agents as indicated, carbon source is 20 g L-1 glucose if not otherwise indicated) for phenotyping and incubated at 300 C for 48 hours. Cells were duplicated on a blank YPD plate (containing no additional stress/agent) for each growth assay to normalize for inherent growth differences. Growth was monitored daily in 1536-spot format by scanning the plates; colony size was quantified by using the programming language R (www.r-project.org) with package R/gitter v1.1.1 (Wagih and Parts, 2014). Prior to data analysis, cell growth was normalized by equating colony sizes of the trait to that of the corresponding blank plate.

### Variant Replacements

Validation of the candidate variants was carried out in two steps. First, the locus harboring a candidate variant was deleted in both parent strains by genomic integration of a disruption cassette containing the nourseothricin (clonNAT) resistance gene (NatMX). The deletion cassette was obtained by PCR from the plasmid pV1382 (addgene, USA) using primers del_QTL_fw and del_QTL_rv (Table 1). When phenotypic difference was observed between the constructed mutant and wild-type strain, the candidate variant was subsequently swapped between the parents via CRISPR-Cas9 mediated genome editing. To target each candidate variant, an unique guide RNA (gRNA_QTN_fw; gRNA_QTN_rv) containing plasmid was constructed based on pV1382 as the backbone (Table 1). Repair fragments (100 bp) containing parental genotype of each target variant was prepared by annealing primers RF_QTN_fw_parent and RF_QTN_rv_parent (Table 1) with 50-60 bp extensions homologous to regions up- and downstream of the target locus. To swap the target QTN in the parent strains, the respective guide RNA plasmid and the repair fragments containing the genotype of the counterparts were co-transformed reciprocally. Transgenic strains were selected on YPD solid medium supplemented with 200 ug mL-1 ClonNAT. The correct constructs of the QTL deletion and QTN swap mutants were verified with PCR and/or Sanger sequencing using primers ver_QTL_fw and ver_QTL_rv (Table 1).

TABLE 1
Sequences used for variant replacements

| Primer name | Sequence |
|---|---|
| del_SUC2_fw | GAAGAAATACGCGTAGCGTTAATCGACCCCACGTCCAGGGTTTTTCCATGCGGGCGAATT |
| | TCTGTCGAGTCATG |
| del_SUC2_rv | AGAATGGCTTTTGAAAAAAATAAAAAAGACAATAAGTTTTATAACCTCTAGCGGCCGCATC |
| | AAGCTTG |
| del_IMA1_fw | TTTGTAGGGTTTCTTCGCACATTATCATTATTATTCTTTGAGAATACTCACGGGCGAATTTCT |
| | GTCGAGTCATG |
| del_IMA1_rv_RM | ATCAAACAAGATACAAACAAAGCTTTTCAACGTAATATTTACTATCGATGGCGGCCGCATC |
| | AAGCTTG |
| del_IMA1_rv_YJM | TCGATGCCATTTGGATCTCACCATTCTACGACTCGCCACAAGATGATATGGCGGCCGCATC |
| | AAGCTTG |
| del_ALD6_fw | ATCAAGAAACATCTTTAACATACACAAACACATACTATCAGAATACAATGCGGGCGAATTT |
| | CTGTCGAGTCATG |
| del_ALD6_rv | ATGAAAGTATTTTGTGTATATGACGGAAAGAAATGCAGGTTGGTACATTAGCGGCCGCAT |
| | CAAGCTTG |
| del_URA5_fw | GTATGAAGGATACACAAAAAAAATAAAGATTAAGAAAGTTATTCAAAATGCGGGCGAATT |
| | TCTGTCGAGTCATG |
| del_URA5_rv | AGATTAATAGTTCTTAAAAGAGATAAATAAATCATTTAATTAAAAAACTGATTTTTAGCGG |
| | CCGCATCAAGCTTG |
| del_URK1_fw | GATAATTTCATACGTTTAATTTCGAACTCGCATTTATTTTATTTATTATGCGGGCGAATTTCT |
| | GTCGAGTCATG |
| del_URK1_rv | TACGTGCACTATTATTTAATTTTACTTTATATTGCCTCTAATTATTCTCAGCGGCCGCATCAA |
| | GCTTG |
| gRNA_SUC2^{-6T>C}_fw | GATCGGCAAGCTTTCCTTTTCCTTTG |
| gRNA_SUC2^{-6T>C}_rv | AAAACAAAGGAAAAGGAAAGCTTGCC |
| gRNA_SUC2^{394fs}_fw | GATCGATGTATTGCTCTTCACTTTCG |
| gRNA_SUC2^{394fs}_rv | AAAACGAAAGTGAAGAGCAATACATC |
| gRNA_ALD6^{184C>A}_fw | GATCGCACTGAAAACACCGTTTGTGG |
| gRNA_ALD6^{184C>A}_rv | AAAACCACAAACGGTGTTTTCAGTGC |
| gRNA_IMA1^{1007A>T}_fw | GATCGACCTTTGTTCCGTTACAACTG |
| gRNA_IMA1^{1007A>T}_rv | AAAACAGTTGTAACGGAACAAAGGTC |
| gRNA_IMA1^{+659G>C}_fw | GATCGTCACAATGTCACAGTCTCAAG |
| gRNA_IMA1^{+659G>C}_rv | AAAACTTGAGACTGTGACATTGTGAC |
| gRNA_URK1^{412A>C}_fw | GATCGAGTCATATACGGGGCCAGTGG |
| gRNA_URK1^{412A>C}_rv | AAAACCACTGGCCCCGTATATGACTC |
| gRNA_URK1^{1358G>A}_fw | GATCGCAAATGCATTTAATATACGTG |
| gRNA_URK1^{1358G>A}_rv | AAAACACGTATATTAAATGCATTTGC |
| RF_SUC2^{-6T>C}_fw_RM | ACAAGCAAAACAAAAAGCTTTTCTTTTCACTAACGTATATGATGCTTTTGCAAGCTTTCC |
| RF_SUC2^{-6T>C}_fw_YJM | ACAAGCAAAACAAAAAGCTTTTCTTTTCACTAACGCATATGATGCTTTTGCAAGCTTTCC |
| RF_SUC2^{6T>C}_rv | GATGCAGATATTTTGGCTGCAAAACCAGCTAAAAGGAAAAGGAAAGCTTGCAAAAGCATC |
| RF_SUC2^{394f}_^{s}fw_RM | TTGATCCAAGACAAAGATGCGTTGCGATTTGGACTTATAACACTCCTGAAAGTGAGGAAC |
| RF_SUC2^{394fs}_fw_YJM | TTGATCCAAGACAAAGATGCGTTGCGATTTGGTTTATAACACTCCTGAAAGTGAGGAAC |
| RF_SUC2^{394fs}_rv | GTAAAAGTGTAACCACCATCAAGAGAATAGCTAATATACTGTTCCTCACTTTCAGGAGTG |
| RF_IMA1^{1007A>T}_fw_RM | TCCAGATAGATTGTTGACCAACAATCTGTACCATTAATGAACCTGAACAGCTCAGCAAGGG |
| | CAATCTTCCAATCCTTCAG |
| RF_IMA1^{1007A>T}_fw_YJM | TCCAGATAGATTGTTGACCAACAATCTGTACCATTAATGTACCTGAACAGCTCAGCAAGGG |
| | CAATCTTCCAATCCTTCAG |
| RF_IMA1^{1007A>T}_rv | GACTTCACCTTTGTTCCGTTACAACTTAGTCCCATTTGAACTGAAGGATTGGAAGATTGC |
| RF_IMA1^{+659G>C}_fw_RM | GAAAGCCATTTTTAATGAGTTATATAGCGTCGTTGATTAGGTATCGTATCACAATGTCAC |
| RF_IMA1^{+659G>C}_fw_YJM | GAAAGCCATTTTTAATGAGTTATATAGGGTCGTTGATTAGGTATCGTATCACAATGTCAC |
| RF_IMA1^{+659G>C}_rv | ACTGGAAGGAGTGATGGTTGATGTATTTTCTCTTGAGACTGTGACATTGTGATACGATAC |
| RF_ALD6^{184C>A}_fw_RM | CGATAGCATATTCAACATCTTCAGGGGTGGCAGAAGAGACTTCACAAACGGTGTTTTCAG |
| RF_ALD6^{184C>A}_fw_YJM | CGATAGCATATTCAACATCTTCAGTGGTGGCAGAAGAGACTTCACAAACGGTGTTTTCAG |
| RF_ALD6^{184C>A}_rv | GCTCAAGACGGTAAGACCTATCCCGTCGAAGATCCTTCCACTGAAAACACCGTTTGTGAA |
| RF_URK1^{412A>C}_fw_RM | ATTTTGAACTTAAAGGAGGGCAAAAGGACAAATATCCCAGTTTATAGCTTCGTCCACCACA |
| | ATAGAGTTCCTGATAAAAA |
| RF_URK1^{412A>C}_fw_YJM | ATTTTGAACTTAAAGGAGGGCAAAAGGACAAATCTCCCAGTTTATAGCTTCGTCCACCACA |
| | ATAGAGTTCCTGATAAAAA |
| RF_URK1^{412A>C}_rv | CGATCGTAAAGGGCGTAGATCCCTTCGATAACTACTACACTGGCCCCGTATATGACTATAT |
| | TTTTATCAGGAACTCTATT |
| RF_URK1^{1358G>A}_fw_RM | TTAGCGTGGTGGTTTATTTGGCCACTGGAGTTGGTATCAGACGTATATTAAATGCATTTG |
| RF_URK1^{1358G>A}_fw_YJM | TTAGCGTGGTGGTTTATTTGGCCACTGAAGTTGGTATCAGACGTATATTAAATGCATTTG |
| RF_URK1^{1358G>A}_rv | CTGGAGATGATCATACCAGCAAAAATGTTGACTTTGTTATCAAATGCATTTAATATACGT |
| ver_SUC2_fw | GCCTTTGTTGAACTCGATCC |
| ver_SUC2_rv | CATAAAGTTTTACATTCGTCACTCG |
| ver_IMA1_fw | AGTATCTACGGCGCAGTAC |
| ver_IMA1_rv | CAGATCAAACAAGATACAAACAAAGC |
| ver_ALD6_fw | GTTTGGTAATATTCAATTCGAAGTG |
| ver_ALD6_rv | GGCTGATGAATTGGAAAGC |
| ver_URA5_fw | TTCCATAAAGCATTACTTCTGCG |
| ver_URA5_rv | GCGTGCATGTATCGTAGTAAC |
| ver_URK1_fw | GAGAGGTGTACCAGCCAG |
| ver_URK1_rv | CCACTTGTTCTCACTATTTCCTC |

### Statistical Analyses

All statistical analyses and graphics were realized using R Core Team (2020), and RStudio Team (2020). R: A language and environment for statistical computing. R Foundation for Statistical Computing, Vienna, Austria. RStudio Team (2020). RStudio: Integrated Development for R. RStudio, PBC, Boston, MA URL http://www.rstudio.com/.

SEQUENCES

SEQ ID No. 1 (SUC2 WT DNA)
ATGCTTTTGCAAGCTTTCCTTTTCCTTTTGGCTGGTTTTGCAGCCAAAATATCTGCATCAAT
GACAAACGAAACTAGCGATAGACCTTTGGTCCACTTCACACCCAACAAGGGCTGGATGAAT
GACCCAAATGGGTTGTGGTACGATGAAAAAGATGCCAAATGGCATCTGTACTTTCAATACA
ACCCAAATGACACCGTATGGGGTACGCCATTGTTTTGGGGCCATGCTACTTCCGATGATTT
GACTCATTGGGAAGATGAACCCATTGCTATCGCTCCCAAGCGTAACGATTCAGGTGCTTTC
TCTGGCTCCATGGTGGTTGATTACAACAACACCAGTGGGTTTTTCAATGATACTATTGATCC
AAGACAAAGATGCGTTGCAATTTGGACTTATAACACTCCTGAAAGTGAAGAGCAATACATT
AGCTATTCCCTTGATGGTGGTTACACTTTTACTGAATACCAAAAGAACCCTGTTTTAGCTGC
CAACTCCACTCAATTCAGAGATCCAAAGGTGTTCTGGTATGAACCTTCTCAAAAATGGATT
ATGACGGCTGCCAAATCACAAGACTACAAAATTGAAATTTACTCCTCGGATGACTTGAAGT
CCTGGAAGTTAGAATCTGCATTTGCCAATGAAGGTTTCTTAGGCTACCAATATGAATGTCC
AGGTTTGATTGAAGTCCCAACTGAGCAAGATCCTTCCAAATCCTATTGGGTCATGTTTATTT
CTATCAACCCAGGTGCACCTGCTGGCGGTTCCTTCAACCAATATTTTGTTGGATCCTTCAAT
GGTACTCATTTTGAAGCGTTTGACAATCAATCTAGAGTGGTAGATTTTGGTAAGGACTACT
ATGCCTTACAAACTTTCTTCAACACTGACCCAACCTACGGTTCAGCATTAGGTATTGCCTGG
GCTTCAAACTGGGAGTACAGTGCCTTTGTCCCAACTAACCCATGGAGATCATCCATGTCTTT
GGTCCGCAAGTTTTCTTTGAACACTGAATATCAAGCTAATCCAGAGACTGAATTGATCAAT
TTGAAAGCCGAACCAATATTGAACATTAGTAATGCTGGCCCCTGGTCTCGTTTTGCTACTA
ACACAACTCTAACTAAGGCCAATTCTTACAATGTCGATTTGAGCAACTCGACTGGTACCCT
AGAGTTTGAGTTGGTTTACGCTGTTAACACCACACAAACCATATCCAAATCCGTCTTCCCCG
ACTTATCACTTTGGTTCAAGGGTTTAGAAGATCCTGAAGAATATTTGAGAATGGGTTTTGA
AGTCAGTGCTTCTTCCTTCTTTTTGGACCGTGGTAACTCTAAGGTCAAGTTTGTCAAGGAGA
ACCCATATTTCACAAACAGAATGTCTGTCAACAACCAACCATTCAAGTCTGAGAACGACCT
AAGTTACTATAAAGTGTACGGCCTACTGGATCAAAACATCTTGGAATTGTACTTCAACGAT
GGAGATGTGGTTTCTACAAATACCTACTTCATGACCACCGGTAACGCTCTAGGATCTGTGA
ACATGACCACCGGTGTCGATAATTTGTTCTACATTGACAAGTTCCAAGTAAGGGAAGTAAA
ATAG
SEQ ID No. 2 (SUC2^{394Δ}; YJM975α DNA)
ATGCTTTTGCAAGCTTTCCTTTTCCTTTTGGCTGGTTTTGCAGCCAAAATATCTGCATCAAT
GACAAACGAAACTAGCGATAGACCTTTGGTCCACTTCACACCCAACAAGGGCTGGATGAAT
GACCCAAATGGGTTGTGGTACGATGAAAAAGATGCCAAATGGCATCTGTACTTTCAATACA
ACCCAAATGACACCGTATGGGGTACGCCATTGTTTTGGGGCCATGCTACTTCCGATGATTT
GACTCATTGGGAAGATGAACCCATTGCTATCGCTCCCAAGCGTAACGATTCAGGTGCTTTC
TCTGGCTCCATGGTGGTTGATTACAACAACACCAGTGGGTTTTTCAATGATACTATTGATCC
AAGACAAAGATGCGTTGCAATTTGGITTATAACACTCCTGAAAGTGAAGAGCAATACATTA
GCTATTCCCTTGATGGTGGTTACACTTTTACTGAATACCAAAAGAACCCTGTTTTAGCTGCC
AACTCCACTCAATTCAGAGATCCAAAGGTGTTCTGGTATGAACCTTCTCAAAAATGGATTA
TGACGGCTGCCAAATCACAAGACTACAAAATTGAAATTTACTCCTCGGATGACTTGAAGTC
CTGGAAGTTAGAATCTGCATTTGCCAATGAAGGTTTCTTAGGCTACCAATATGAATGTCCA
GGTTTGATTGAAGTCCCAACTGAGCAAGATCCTTCCAAATCCTATTGGGTCATGTTTATTTC
TATCAACCCAGGTGCACCTGCTGGCGGTTCCTTCAACCAATATTTTGTTGGATCCTTCAATG
GTACTCATTTTGAAGCGTTTGACAATCAATCTAGAGTGGTAGATTTTGGTAAGGACTACTA
TGCCTTACAAACTTTCTTCAACACTGACCCAACCTACGGTTCAGCATTAGGTATTGCCTGGG
CTTCAAACTGGGAGTACAGTGCCTTTGTCCCAACTAACCCATGGAGATCATCCATGTCTTTG
GTCCGCAAGTTTTCTTTGAACACTGAATATCAAGCTAATCCAGAGACTGAATTGATCAATTT
GAAAGCCGAACCAATATTGAACATTAGTAATGCTGGCCCCTGGTCTCGTTTTGCTACTAAC
ACAACTCTAACTAAGGCCAATTCTTACAATGTCGATTTGAGCAACTCGACTGGTACCCTAG
AGTTTGAGTTGGTTTACGCTGTTAACACCACACAAACCATATCCAAATCCGTCTTCCCCGAC
TTATCACTTTGGTTCAAGGGTTTAGAAGATCCTGAAGAATATTTGAGAATGGGTTTTGAAG
TCAGTGCTTCTTCCTTCTTTTTGGACCGTGGTAACTCTAAGGTCAAGTTTGTCAAGGAGAAC
CCATATTTCACAAACAGAATGTCTGTCAACAACCAACCATTCAAGTCTGAGAACGACCTAA
GTTACTATAAAGTGTACGGCCTACTGGATCAAAACATCTTGGAATTGTACTTCAACGATGG
AGATGTGGTTTCTACAAATACCTACTTCATGACCACCGGTAACGCTCTAGGATCTGTGAAC
ATGACCACCGGTGTCGATAATTTGTTCTACATTGACAAGTTCCAAGTAAGGGAAGTAAAAT
AG
SEQ ID No. 3 (SUC2 WT protein)
MLLQAFLFLLAGFAAKISASMTNETSDRPLVHFTPNKGWMNDPNGLWYDEKDAKWHLYFQ
YNPNDTVWGTPLFWGHATSDDLTHWEDEPIAIAPKRNDSGAFSGSMVVDYNNTSGFFNDTI
DPRQRCVAIWTYNTPESEEQYISYSLDGGYTFTEYQKNPVLAANSTQFRDPKVFWYEPSQKW
IMTAAKSQDYKIEIYSSDDLKSWKLESAFANEGFLGYQYECPGLIEVPTEQDPSKSYWVMFISI
NPGAPAGGSFNQYFVGSFNGTHFEAFDNQSRVVDFGKDYYALQTFFNTDPTYGSALGIAWA
SNWEYSAFVPTNPWRSSMSLVRKFSLNTEYQANPETELINLKAEPILNISNAGPWSRFATNTT
LTKANSYNVDLSNSTGTLEFELVYAVNTTQTISKSVFPDLSLWFKGLEDPEEYLRMGFEVSASS
FFLDRGNSKVKFVKENPYFTNRMSVNNQPFKSENDLSYYKVYGLLDQNILELYFNDGDVVST
NTYFMTTGNALGSVNMTTGVDNLFYIDKFQVREVK*
SEQ ID No. 4 (SUC2^{394Δ}; YJM975α protein)
MLLQAFLFLLAGFAAKISASMTNETSDRPLVHFTPNKGWMNDPNGLWYDEKDAKWHLYFQ
YNPNDTVWGTPLFWGHATSDDLTHWEDEPIAIAPKRNDSGAFSGSMVVDYNNTSGFFNDTI
DPRQRCVAIWFITLLKVKSNTLAIPLMVVTLLLNTKRTLF*
SEQ ID No. 5 (URK1 WT DNA)
ATGTCCCATCGTATAGCACCTTCCAAAGAACGATCTTCATCATTTATTTCAATTTTAGACGA
TGAAACAAGAGACACATTGAAAGCTAATGCAGTCATGGATGGTGAAGTAGATGTCAAAAA
AACAAAAGGAAAAAGCTCTCGGTATATCCCACCATGGACAACTCCATATATAATAGGTATA
GGTGGTGCTTCAGGTTCAGGCAAGACAAGCGTTGCTGCTAAGATTGTGTCGTCAATTAATG
TTCCCTGGACAGTATTAATATCTTTGGATAACTTTTACAATCCATTAGGCCCAGAGGACAG
AGCCAGAGCCTTTAAAAATGAATACGATTTCGACGAGCCAAATGCCATCAACTTAGATTTG
GCATATAAGTGCATTTTGAACTTAAAGGAGGGCAAAAGGACAAATATCCCAGTTTATAGCT
TCGTCCACCACAATAGAGTTCCTGATAAAAATATAGTCATATACGGGGCCAGTGTGGTAGT
TATCGAAGGGATCTACGCCCTTTACGATCGCCGATTGCTGGATTTGATGGACTTGAAAATT
TATGTTGACGCTGATTTGGATGTCTGCTTAGCAAGAAGATTGTCGAGAGATATAGTTTCCA
GAGGGAGAGATTTGGATGGTTGTATTCAACAATGGGAGAAATTTGTGAAACCAAATGCGG
TAAAGTTTGTGAAACCAACAATGAAGAATGCAGATGCTATCATTCCATCGATGAGTGATAA
TGCTACAGCGGTAAATTTAATCATTAACCACATCAAGTCAAAACTGGAACTAAAATCAAAT
GAACACTTAAGAGAGCTAATCAAATTGGGCTCTTCTCCTTCACAAGATGTGCTTAATCGTA
ACATAATTCATGAATTGCCGCCCACCAACCAAGTTCTTTCGCTGCATACTATGCTTCTAAAT
AAAAATCTAAATTGCGCGGACTTTGTTTTCTACTTTGACAGGTTAGCAACAATTTTGTTATC
ATGGGCACTTGATGACATTCCTGTAGCACATACGAACATAATTACACCTGGTGAGCATACC
ATGGAAAACGTTATTGCCTGTCAATTCGATCAAGTTACAGCTGTTAATATTATTCGATCTGG
CGATTGTTTTATGAAGTCTTTGAGAAAGACGATCCCCAATATCACAATTGGTAAATTGTTG
ATTCAGTCCGATTCACAAACTGGGGAACCTCAACTGCATTGCGAATTTTTACCCCCAAATAT
AGAAAAGTTTGGCAAGGTTTTCTTAATGGAAGGTCAAATCATAAGTGGTGCGGCCATGATC
ATGGCCATCCAGGTGCTTTTAGATCATGGTATTGATTTGGAAAAGATTAGCGTGGTGGTTT
ATTTGGCCACTGGAGTTGGTATCCGACGTATATTAAATGCATTTGATAACAAAGTCAACAT
TTTTGCTGGTATGATCATCTCCAGAGAAAAGTTACAAAATCATCAATACAAATGGGCATTG
ACCAGATTTCTTGATTCAAAGTATTTTGGTTGTGATTGA
SEQ ID No. 6 (IMA1 WT DNA)
ATGACTATTTCTTCTGCACATCCAGAGACAGAACCAAAGTGGTGGAAAGAGGCCACGTTCT
ATCAAATTTACCCAGCAAGTTTCAAAGACTCTAATGACGATGGCTGGGGTGATATGAAGG
GTATTTCCTCCAAGTTGGAGTATATCAAGGAGCTTGGTGTCGATGCCATTTGGATCTCACC
ATTCTACGACTCGCCACAAGATGATATGGGTTACGATATTGCCAACTACGAAAAGGTCTGG
CCAACCTACGGTACGAACGAGGACTGCTTTGCCTTGATCGAAAAGACACATAAACTTGGTA
TGAAATTTATCACCGACTTGGTCATCAATCACTGTTCCAGCGAACATGAATGGTTCAAAGA
GAGCAGATCCTCGAAGACCAATCCGAAGCGTGACTGGTTCTTCTGGAGACCTCCTAAGGGT
TATGACGCCGAAGGCAAGCCAATTCCTCCAAATAATTGGAAGTCCTATTTTGGTGGTTCCG
CATGGACCTTCGATGAAAAGACACAAGAATTCTACTTGCGTTTGTTTTGCTCCACTCAACCT
GATTTGAATTGGGAGAATGAAGACTGTAGAAAGGCAATCTACGAAAGTGCCGTTGGATAC
TGGTTAGACCATGGTGTAGACGGCTTTAGAATTGATGTCGGAAGTTTGTACTCCAAAGTTG
TAGGTTTACCAGATGCCCCTGTTGTTGACAAAAACTCGACTTGGCAATCCAGTGATCCATG
CACATTGAATGGACCACGTATTCACGAGTTCCATCAAGAAATGAATCAATTCATCAGAAAC
AGAGTGAAGGATGGCAGGGAGATTATGACAGTTGGTGAAATGCAACATGCTTCCGACGAA
ACTAAGAGACTTTATACGAGTGCTTCAAGACACGAACTTAGTGAGTTATTTAACTTTTCCCA
CACTGATGTGGGGACTTCACCTTTGTTCCGTTACAACTTGGTCCCATTTGAACTGAAGGATT
GGAAGATTGCCCTTGCTGAGCTGTTCAGGTTCATTAATGGTACAGATTGTTGGTCAACAAT
CTATCTGGAAAATCACGACCAACCTCGTTCAATTACGAGATTTGGTGACGATTCTCCTAAG
AACCGTGTTATTTCTGGTAAGTTACTCTCTGTGTTGCTAAGTGCCTTGACCGGTACTCTATA
TGTGTATCAGGGACAAGAGCTTGGCCAAATCAATTTCAAGAACTGGCCTGTTGAAAAGTAC
GAGGATGTCGAAATCAGAAACAACTACAATGCAATTAAAGAAGAGCATGGGGAAAACTCA
GAGGAGATGAAAAAGTTTTTAGAAGCCATTGCCCTTATCTCCAGGGACCATGCTAGAACAC
CTATGCAATGGTCTCGTGAGGAGCCAAATGCTGGTTTTTCTGGTCCTAGTGCTAAACCATG
GTTTTACTTGAACGACTCTTTCAGAGAAGGCATTAACGTCGAAGATGAAATCAAGGATCCC
AACTCGGTTTTGAACTTCTGGAAGGAGGCCTTGAAGTTTAGAAAGGCGCATAAAGACATTA
CTGTGTACGGATACGATTTCGAGTTTATTGATTTAGACAATAAGAAGTTGTTTAGCTTCACA
AAGAAGTACAACAATAAAACATTGTTTGCGGCTTTGAACTTTAGCTCTGATGCGACAGATT
TCAAGATTCCAAATGATGATTCATCGTTCAAGTTAGAGTTTGGAAACTATCCAAAGAAGGA
GGTAGATGCCTCTTCCAGAACATTGAAGCCATGGGAAGGAAGAATATATATCAGCGAATG
A
SEQ ID No. 7 (ALD6 WT DNA)
ATGACTAAGCTACACTTTGACACTGCTGAACCAGTCAAGATCACACTTCCAAATGGTTTGA
CATACGAGCAACCAACCGGTCTATTCATTAACAACAAGTTTATGAAAGCTCAAGACGGTAA
GACCTATCCCGTCGAAGATCCTTCCACTGAAAACACCGTTTGTGAGGTCTCTTCTGCCACCA
CTGAAGATGTTGAATATGCTATCGAATGTGCCGACCGTGCTTTCCACGACACTGAATGGGC
TACCCAAGACCCAAGAGAAAGAGGCCGTCTACTAAGTAAGTTGGCTGATGAATTGGAAAG
CCAAATTGACTTGGTTTCTTCCATTGAAGCTTTGGACAATGGTAAAACTTTGGCCTTAGCCC
GTGGGGATGTTACCATTGCAATCAACTGTCTAAGAGATGCTGCTGCCTATGCCGACAAAGT
CAACGGTAGAACAATCAACACCGGTGACGGCTACATGAACTTCACCACCTTAGAGCCAATC
GGTGTCTGTGGTCAAATTATTCCATGGAACTTTCCAATAATGATGTTGGCTTGGAAGATCG
CCCCAGCATTGGCCATGGGTAACGTCTGTATCTTGAAACCCGCTGCTGTCACACCTTTAAAT
GCCCTATACTTTGCTTCTTTATGTAAGAAGGTTGGTATTCCAGCTGGTGTCGTCAACATCGT
TCCAGGTCCTGGTAGAACTGTTGGTGCTGCTTTGACCAACGACCCAAGAATCAGAAAGCTG
GCTTTTACCGGTTCTACGGAAGTCGGTAAGAGTGTTGCTGTCGACTCTTCTGAATCTAACTT
GAAGAAAATCACTTTGGAACTAGGTGGTAAGTCCGCCCATTTGGTCTTTGACGATGCTAAC
ATTAAGAAGACTTTACCAAATCTAGTAAACGGTATCTTCAAGAACGCTGGTCAAATTTGTT
CCTCTGGTTCTAGAATTTACGTTCAAGAAGGTATTTACGACGAACTATTGGCTGCTTTCAAG
GCTTACTTGGAAACCGAAATCAAAGTTGGTAATCCATTTGACAAGGCTAACTTCCAAGGTG
CTATCACTAACCGTCAACAATTCGACACAATTATGAACTACATCGATATCGGTAAGAAAGA
AGGCGCCAAGATCTTAACTGGTGGCGAAAAAGTTGGTGACAAGGGTTACTTCATCAGACC
AACCGTTTTCTACGATGTTAATGAAGACATGAGAATTGTTAAGGAAGAAATTTTTGGACCA
GTTGTCACTGTCGCAAAGTTCAAGACTTTAGAAGAAGGTGTCGAAATGGCTAACAGCTCTG
AATTCGGTCTAGGTTCTGGTATCGAAACAGAATCTTTGAGCACAGGTTTGAAGGTGGCCAA
GATGTTGAAGGCCGGTACCGTCTGGATCAACACATACAACGATTTTGACTCCAGAGTTCCA
TTCGGTGGTGTTAAGCAATCTGGTTACGGTAGAGAAATGGGTGAAGAAGTCTACCATGCA
TACACTGAAGTAAAAGCTGTCAGAATTAAGTTGTAA
SEQ ID No. 8 (ALD6^{184A>C}; RM11-1a DNA)
ATGACTAAGCTACACTTTGACACTGCTGAACCAGTCAAGATCACACTTCCAAATGGTTTGA
CATACGAGCAACCAACCGGTCTATTCATTAACAACAAGTTTATGAAAGCTCAAGACGGTAA
GACCTATCCCGTCGAAGATCCTTCCACTGAAAACACCGTTTGTGAGGTCTCTTCTGCCACCC
CTGAAGATGTTGAATATGCTATCGAATGTGCCGACCGTGCTTTCCACGACACTGAATGGGC
TACCCAAGACCCAAGAGAAAGAGGCCGTCTACTAAGTAAGTTGGCTGATGAATTGGAAAG
CCAAATTGACTTGGTTTCTTCCATTGAAGCTTTGGACAATGGTAAAACTTTGGCCTTAGCCC
GTGGGGATGTTACCATTGCAATCAACTGTCTAAGAGATGCTGCTGCCTATGCCGACAAAGT
CAACGGTAGAACAATCAACACCGGTGACGGCTACATGAACTTCACCACCTTAGAGCCAATC
GGTGTCTGTGGTCAAATTATTCCATGGAACTTTCCAATAATGATGTTGGCTTGGAAGATCG
CCCCAGCATTGGCCATGGGTAACGTCTGTATCTTGAAACCCGCTGCTGTCACACCTTTAAAT
GCCCTATACTTTGCTTCTTTATGTAAGAAGGTTGGTATTCCAGCTGGTGTCGTCAACATCGT
TCCAGGTCCTGGTAGAACTGTTGGTGCTGCTTTGACCAACGACCCAAGAATCAGAAAGCTG
GCTTTTACCGGTTCTACGGAAGTCGGTAAGAGTGTTGCTGTCGACTCTTCTGAATCTAACTT
GAAGAAAATCACTTTGGAACTAGGTGGTAAGTCCGCCCATTTGGTCTTTGACGATGCTAAC
ATTAAGAAGACTTTACCAAATCTAGTAAACGGTATCTTCAAGAACGCTGGTCAAATTTGTT
CCTCTGGTTCTAGAATTTACGTTCAAGAAGGTATTTACGACGAACTATTGGCTGCTTTCAAG
GCTTACTTGGAAACCGAAATCAAAGTTGGTAATCCATTTGACAAGGCTAACTTCCAAGGTG
CTATCACTAACCGTCAACAATTCGACACAATTATGAACTACATCGATATCGGTAAGAAAGA
AGGCGCCAAGATCTTAACTGGTGGCGAAAAAGTTGGTGACAAGGGTTACTTCATCAGACC
AACCGTTTTCTACGATGTTAATGAAGACATGAGAATTGTTAAGGAAGAAATTTTTGGACCA
GTTGTCACTGTCGCAAAGTTCAAGACTTTAGAAGAAGGTGTCGAAATGGCTAACAGCTCTG
AATTCGGTCTAGGTTCTGGTATCGAAACAGAATCTTTGAGCACAGGTTTGAAGGTGGCCAA
GATGTTGAAGGCCGGTACCGTCTGGATCAACACATACAACGATTTTGACTCCAGAGTTCCA
TTCGGTGGTGTTAAGCAATCTGGTTACGGTAGAGAAATGGGTGAAGAAGTCTACCATGCA
TACACTGAAGTAAAAGCTGTCAGAATTAAGTTGTAA
SEQ ID No. 9 (ALD6 WT protein)
MTKLHFDTAEPVKITLPNGLTYEQPTGLFINNKFMKAQDGKTYPVEDPSTENTVCEVSSATTE
DVEYAIECADRAFHDTEWATQDPRERGRLLSKLADELESQIDLVSSIEALDNGKTLALARGDV
TIAINCLRDAAAYADKVNGRTINTGDGYMNFTTLEPIGVCGQIIPWNFPIMMLAWKIAPALAM
GNVCILKPAAVTPLNALYFASLCKKVGIPAGVVNIVPGPGRTVGAALTNDPRIRKLAFTGSTEV
GKSVAVDSSESNLKKITLELGGKSAHLVFDDANIKKTLPNLVNGIFKNAGQICSSGSRIYVQEGI
YDELLAAFKAYLETEIKVGNPFDKANFQGAITNRQQFDTIMNYIDIGKKEGAKILTGGEKVGDK
GYFIRPTVFYDVNEDMRIVKEEIFGPVVTVAKFKTLEEGVEMANSSEFGLGSGIETESLSTGLKV
AKMLKAGTVWINTYNDFDSRVPFGGVKQSGYGREMGEEVYHAYTEVKAVRIKL*
SEQ ID No. 10 (ALD6^{T62P}; RM11-1a protein)
MTKLHFDTAEPVKITLPNGLTYEQPTGLFINNKFMKAQDGKTYPVEDPSTENTVCEVSSATPE
DVEYAIECADRAFHDTEWATQDPRERGRLLSKLADELESQIDLVSSIEALDNGKTLALARGDV
TIAINCLRDAAAYADKVNGRTINTGDGYMNFTTLEPIGVCGQIIPWNFPIMMLAWKIAPALAM
GNVCILKPAAVTPLNALYFASLCKKVGIPAGVVNIVPGPGRTVGAALTNDPRIRKLAFTGSTEV
GKSVAVDSSESNLKKITLELGGKSAHLVFDDANIKKTLPNLVNGIFKNAGQICSSGSRIYVQEGI
YDELLAAFKAYLETEIKVGNPFDKANFQGAITNRQQFDTIMNYIDIGKKEGAKILTGGEKVGDK
GYFIRPTVFYDVNEDMRIVKEEIFGPVVTVAKFKTLEEGVEMANSSEFGLGSGIETESLSTGLKV
AKMLKAGTVWINTYNDFDSRVPFGGVKQSGYGREMGEEVYHAYTEVKAVRIKL*

### REFERENCES

Liti et al 2009 Nature 458(7236): 337-341.
Fay & Benavides 2005 PLOS Genet
Carreto et al 2008 BMC Genomics 9:1-17.
Gallone et al 2019 Nat Ecol Evol 3(11):1562-1575.
Gallone et al 2016 Cell 166(6):1397-1410.
She & Jarosz 2018 Cell 172(3):478-490.
Offei et al 2019 Genome Res 29(9):1478-1494.
Hoffman & Winston 1987 Gene 57(2-3):267-272.
Steensels et al. 2014 Appl Environ Microbiol 80(22):6965-6975.
Wagih & Parts 2014 G3 (Bethesda) 2014 Jan. 28; 4.

1. An industrial yeast strain comprising:
a disrupted, partially deleted or completely deleted ALD6 allele; or
a chimeric gene construct comprising a promoter active in yeast operably linked to a Crispr guide RNA targeting an ALD6 allele.

2. The industrial yeast strain of claim 1, wherein the disrupted, partially deleted or completely deleted ALD6 allele is present in homozygous or hemizygous form.

3. The industrial yeast strain of claim 1, wherein the ALD6 allele is disrupted or partially deleted and encodes an Ald6 protein comprising a T to P mutation on position 62 of SEQ ID No. 9.

4. The industrial yeast strain according to any of preceding claims, wherein the disrupted, partially deleted or completely deleted ALD6 allele encodes SEQ ID No. 10.

5. The industrial yeast strain of claim 1, wherein the yeast is Saccharomyces cerevisiae.

6. The industrial yeast strain of claim 1, wherein the yeast is S. cerevisiae var. boulardii.

7. A chimeric gene construct comprising a promoter active in yeast operably linked to a Crispr guide RNA targeting an ALD6 allele.

8. (canceled)

9. (canceled)

10. (canceled)

11. (canceled)

12. (canceled)

13. A method of increasing acetic acid production in a yeast fermentation comprising administering the yeast strain of claim 1 to a fermentation medium and optionally determining the level of acetic acid.

14. (canceled)

15. A method the treatment of gastrointestinal disorders, diarrhea, gastrointestinal discomfort and/or constipation in a subject, the method comprising: administering to the subject the industrial yeast strain of claim 6.

16. The industrial yeast strain of claim 6, wherein the industrial yeast strain is comprised in a food or feed product, beverage, food supplement, dietary supplement or pharmaceutical composition.

17. A chimeric gene construct comprising a promoter active in yeast operably linked to a Crispr guide RNA targeting a SUC2, IMA1 or URK1 allele.

18. An industrial yeast strain comprising:
a disrupted, partially deleted or completely deleted SUC2, IMA1 or URK1 allele; or
a chimeric gene construct comprising a promoter active in yeast operably linked to a Crispr guide RNA targeting a SUC2, IMA1 or URK1 allele.

19. The industrial yeast strain of claim 18, wherein the disrupted, partially deleted or completely deleted SUC2, IMA1 or URK1 allele is present in homozygous or hemizygous form.

20. The industrial yeast strain of claim 18, wherein the SUC2 allele encodes a truncated Suc2 protein.

21. The industrial yeast strain of claim 18, wherein the SUC2 allele encodes SEQ ID No. 4.

22. (canceled)

23. (canceled)

24. (canceled)

25. A method of reducing the production of ethanol, 1-propanol, ethyl acetate, acetic acid and/or glycerol in a yeast fermentation, the method comprising: administering the industrial yeast strain of claim 18 to a fermentation medium and optionally determining the level of ethanol, 1-propanol, ethyl acetate, acetic acid and/or glycerol.

26. (canceled)

27. A method for reducing the production of glycerol and/or acetic acid or for increasing the production of isobutanol in a yeast fermentation, the method comprising: administering to a fermentation medium a genetic inhibitor of IMA1 or the industrial yeast strain of claim 18.

28. The method according to claim 27, wherein the genetic inhibitor is a disrupted, partially deleted or completely deleted IMA1 allele, a nuclease, a Crispr-Cas effector, the chimeric gene construct comprising a promoter active in yeast operably linked to a Crispr guide RNA targeting a SUC2, IMA1 or URK1 allele or an RNA-silencing agent.

29. The method according to claim 27, further comprising determining the level of glycerol, acetic acid and/or isobutanol.

30. (canceled)

31. (canceled)

32. (canceled)

33. (canceled)
